(19) United States
(12) Patent Application Publication
(10) Pub. No.: US 2021/0155597 A1
(43) Pub. Date: May 27, 2021

(54) COMPOSITIONS AND METHODS FOR TREATMENT OF PAIN

(71) Applicant: SYNVENTA LLC, Tucson, AZ (US)

(72) Inventors: Artour Gomtsian, Tucson, AZ (US); Anton Bespalov, Heidelberg (DE)

(21) Appl. No.: 17/100,433

(22) Filed: Nov. 20, 2020

Related U.S. Application Data
(60) Provisional application No. 62/938,697, filed on Nov. 21, 2019.

(51) Int. Cl. C07D 279/06; C07D 417/12; C07D 487/10; C07D 231/56; C07D 413/04; C07D 405/12; C07D 401/04; C07D 231/06; C07D 231/12; C07C 279/22; A61P 25/00

(52) U.S. Cl. CPC C07D 279/06; C07D 417/12; C07D 487/10; C07D 231/56; C07D 413/04; A61P 25/00; C07D 401/04; C07D 231/06; C07D 231/12; C07C 279/22; C07D 405/12

(57) ABSTRACT

The present disclosure relates to compositions comprising a transient receptor potential vanilloid-1 (TRPV1) antagonist and an alpha-2 adrenoreceptor agonist useful in the treatment of various forms of pain, including chronic pain (CP) syndromes, inflammatory pain and pain associated with neuropathy and other diseases and disease states; and methods of use thereof.

### PRIORITY APPLICATION

This application claims priority to U.S. Application No. 62/938,697, filed Nov. 21, 2019, which is hereby incorporated by reference in its entirety.

### BACKGROUND OF THE INVENTION

Despite significant investment and scientific investigation, a safe and efficacious treatment of pain remains elusive. Existing pain medications suffer from one or more of the following liabilities: narrow safety window, high abuse potential, and reduced efficacy over time.

Transient receptor potential vanilloid-1 (TRPV1) receptor, a member of the vanilloid receptor family, is a nonselective cation channel that is primarily expressed on sensory neurons and is upregulated and/or activated by various noxious stimuli including capsaicin. Antagonism of TRPV1 as a potential therapeutic mechanism that once showed promise has fallen out of favor. Small molecule therapeutics investigated in clinical settings have failed due to either a lack of efficacy and/or toxicity, primarily related to hyperthermia. At least a dozen major pharmaceutical companies have discontinued clinical TRPV1 antagonists. See Peppin, J. F. et. al, Therapeutic Advances in Neurological Disorders, 2014, 7(1): 22-32.

AMG 517 (N-(4-((6-(4-(trifluoromethyl)phenyl)pyrimidin-4-yl)oxy)benzo[d]thiazol-2-yl)acetamide) was developed by Amgen as a TRPV1 antagonist. A Phase 1b study of AMG 517 was “halted due to unexpectedly high temperature elevations,” which was caused “via an unknown mechanism.” 2007 Jan. 30 Amgen Public Results Redacted (available at http://www.amgentrials.com/amgen/studylist.aspx?productid=20). Amgen further reported: “A review of the data for the 17 subjects enrolled indicated no clear analgesic effect.” Id. Based on these findings, the program was terminated. Id.

AZD-1386 (5′-chloro-1′-(3-fluorobenzyl)-7′-methylspiro[imidazolidine-4,3′-indoline]-2,2′,5-trione) was developed by AstraZeneca as a TRPV1 antagonist. Based on the interim analysis of study NCT00878501 it was recommended to stop the study for futility since AZD-1386 showed no significant pain decrease based on the primary variable. Hyperthermia (maximal 1.2° C.) was also observed with AZD-1386. L Stahle, et. al., 13^{th} World Congress on Pain, Montreal, Canada, 2010. Subsequent studies of AZD-1386 were terminated based on the findings in NCT00878501.

Analgesic potential of ABT-102 (1-[(R)-5-tert-butyl-2,3-dihydro-1H-inden-1-yl]-3-(1H-indazol-4-yl)urea) was assessed in an experimental pain study in healthy volunteers (Schaffler, K. et al., Br J Clin Pharmacol. 2013, 75, 404). Again, hyperthermia (0.6-0.8° C.) was observed at the efficacious dose.

In the course of characterizing analgesic properties of structurally distinct TRPV1 antagonists, multiple investigators have observed core body temperature-elevating (“hyperthermic”) attributes of these compounds in rodent behavioral models of pain (Swanson, D. M. et al., J. Med. Chem., 2005, 48, 1857; Gavva, N. R. et al., J. Pharmacol. Exp. Ther, 2007, 323, 128; Steiner, A. A. et al., J. Neurosci., 2007, 27, 7459; Tamayo, N. et al., J. Med. Chem., 2008, 51, 2744; Gavva, N. R. et al., J. Neurosci., 2007, 27, 3366). Often modest (0.5° C.), the associated temperature elevation can be considerably more robust (1-2° C.), and also has been reported preclinically in dogs and monkeys (Gavva, N. R. et al., J. Pharmacol. Exp. Ther., 2007, 323, 128; Gavva, N. R. et al., J. Neurosci., 2007, 27, 3366) and in human subjects in the course of clinical trials (Gavva, N. R. et al., Pain, 2008, 136, 202). These effects have the potential to be self-limiting; they are generally transient and attenuate with repeat dosing (Gavva, N. R. et al., J. Pharmacol. Exp. Ther., 2007, 323, 128). The temperature effects are considered to be mechanism based (Lida, T. et al., Neurosci. Lett., 2005, 378, 28) since TRPV1 null mice show no deficits in thermoregulation, even when dosed with antagonists that elevate temperature in wild-type mice (Steiner, A. A. et al., J. Neurosci., 2007, 27, 7459; Garami, A. et al., J. Neurosci., 2010, 30, 1435).

Recognizing that an increase in body temperature is an adverse event that may preclude further investigation and treatment in humans, others have sought to find a solution to counteract the hyperthermic effect of TRPV1 antagonism by co-administering anti-pyretic agents. See, US2006/0281718. While anti-pyretic agents generally reduce body temperature based on anti-inflammatory pathways, the hyperthermic effect caused by TRPV1 antagonism is an effect outside the anti-inflammatory pathways. Accordingly, administration of anti-pyretic agents with TRPV1 antagonists do not generally mitigate the hyperthermic effect of TRPV1 antagonism.

Modulation of blood pressure can also impact pain conditions. The cardiovascular and pain regulatory systems are structurally and functionally intertwined. In healthy individuals, these interconnections produce blood pressure (BP)-related hypoalgesia, in which elevated BP triggers reduced pain sensation. Although some work suggests BP-related hypoalgesia in some cases may remain intact in individuals with CP, multiple other studies indicate that CP is associated with reduced magnitude of BP-related hypoalgesia, suggesting possible CP-related impairments in cardiovascular regulatory systems potentially relevant to comorbid cardiovascular risk in CP. Population studies indicate that CP is linked to increased hypertension risk. Intensity of chronic pain is a significant predictor of hypertensive status, independent of the effects of age, race, ethnicity, and parental hypertension. Thus, chronic pain, and severity thereof, is associated with an increased risk of hypertension.

In patients with chronic pain (e.g. chronic low back or orofacial pain), elevated BP levels at rest are associated with an increased sensitivity to acute pain and a higher intensity of chronic pain. A positive relationship between resting BP and clinical chronic pain intensity has been observed. The pain regulatory dysfunction reflected in this positive BP-chronic pain relationship is progressive, in relation to the duration of pain.

Therefore, a medical need exists for a safe and effective composition for the treatment of pain. More particularly, a medical need exists for a composition comprising a TRPV1 antagonist that avoids the complications observed in the prior art, namely, that (1) attenuates and/or eliminates the known hyperthermic effect of certain TRPV1 antagonists; (2) increases the efficacy of the TRPV1 antagonist such that a lower dose may achieve the same efficacy; and (3) has little to no effect on arterial blood pressure.

α2-Adrenoceptors are known to be implicated in pain modulation at both peripheral and central sites of the pain processing system. α2-adrenoceptors are expressed in dorsal root ganglion (DRG) neurons and mediate effects of noradrenaline on TRPV1 activity. α2-adrenoceptors have been reported to reduce the activity of TRPV1 in DRG neurons, and this effect is caused by the potentiation of calmodulin-dependent kinase II (CAMKII) activity (Matsushita Y. et al., PLoS ONE, 2018, 13, 1). Moreover, this action of α2-adrenoceptors was demonstrated to reduce capsaicin-evoked neurotransmitter release from the spinal terminals of primary sensory neurons, and this phenomenon was proposed to play a role in the analgesic action of the adrenergic system.

The information as described in the above background art is only provided to more understand a background technology of the present disclosure, however, other information with respect to prior art well known to persons of ordinary skill in the art to which the present disclosure pertains, may not be included herein.

### SUMMARY OF THE INVENTION

The present inventors have surprisingly discovered that a composition comprising a TRPV1 antagonist and an alpha-2 adrenoreceptor agonist (also known as an alpha-2 agonist) has beneficial properties relative to what would be expected as a treatment for pain conditions while avoiding the known adverse effects associated with TRPV1 antagonism.

Various embodiments of the disclosure relate to treatment of pain, CP syndromes, inflammatory pain, cancer pain and pain associated with neuropathy and other diseases, disease conditions and co-morbidities such as those characterized by arterial hypertension and/or hyperthermia with novel compositions, combinations, therapeutic formulations, therapies, kits thereof. For example, the pain condition treated by the composition or compound(s) of the present disclosure include, but are not limited to, acute pain, neuropathic pain, cancer pain, dental pain, pain associated with an inflammatory bowel disorder, inflammatory pain, pain associated with an inflammatory eye disorder, skin pain associated with inflammation and pain associated with hyperalgesia or allodynia. Pain associated with hyperalgesia or allodynia is typically caused by a disease or disease state as reported in the literature, a medication (such as a small molecule), a therapeutic (such as a biologic or combination or medications); an illicit drug, or a recreational drug.

In various embodiments, the invention is a composition comprising a one or more agents, each having a unique Therapeutic Mode of Action (TMA), wherein the agent is TRPV1 receptor antagonist, and/or α2-adrenoceptor agonist.

The present disclosure also describes a method of controlling pain in mammals with a combination of agents comprising a TRPV1 antagonist and known α2-adrenoceptor agonist. In certain embodiments, the TRPV1 antagonist is known to induce hyperthermia.

An embodiment of the invention is a composition comprising a compound of Formula (I)

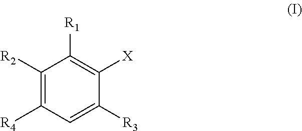

or a pharmaceutically acceptable salt or prodrug thereof, wherein

### R₁ is H, Me or Cl;

### R₂ is H or Me; or

R₁ and R₂ together form

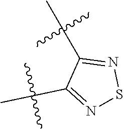 [SMILES: *c1nsnc1*]

### R₃ is H, Me or Cl

R₄ is H or tert-Bu
X is selected from the group consisting of

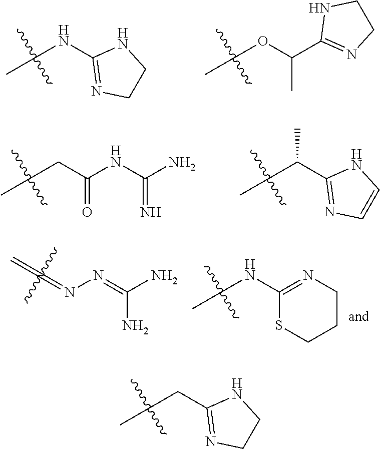

An embodiment of the invention is a composition comprising a compound of Formula (II)

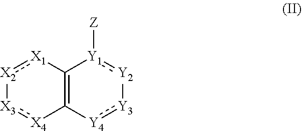

or pharmaceutically acceptable salt or prodrug thereof, wherein [symbol]
is absent or single bond

### X₁ is CH, CMe, N or O;

X₂ is CH, CH₂, CNHAc or N;
X₃ is CR₁, (S)—CHCH₂OH, N, NH, or S;
X₄ is bond, CH or NR₂;

### R₁ is

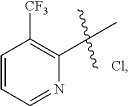 [SMILES: *c1ncccc1C(F)(F)F]

### R₂ is

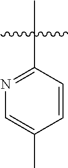 [SMILES: *c1ccc(C)cn1]

### Y₁ is C or N;

### Y₂ is CH, C═O or N;

Y₃ is CH or C-G, where G is a spiro-ring

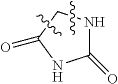

Y₄ is bond, CH or N;

### Z is

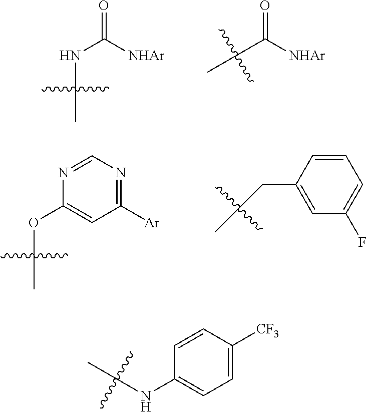

### Ar is

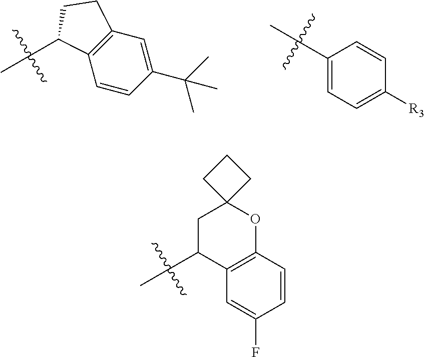

R₃ is CF₃ and OCH₂CF₃. Compounds of Formula (II) are known to increase body temperature after administration.

Some embodiments include a method of treating a disease or disorder in a subject in need thereof comprising an effective amount of: 1) a composition comprising a compound of Formula (I), as defined above; enantiomers thereof, metabolites thereof, derivatives thereof, and/or prodrugs thereof, pharmaceutically acceptable salts thereof, or a combination thereof; and 2) a compound of Formula (II), as defined above, enantiomers, metabolites, derivatives, prodrugs, salts, diastereomers, pharmaceutically acceptable salts, or a combination thereof.

In an embodiment, the method is a method of decreasing the number of doses and/or total daily dose of the compound of Formula (I) that can be administered while increasing efficacy and safeguarding tolerability and safety; a method of reducing an adverse event associated with treatment by the compound of Formula (I), wherein the subject is at risk of experiencing the adverse event as a result being treated with the compound of Formula (I).

In an embodiment, the method is a method of decreasing the number of doses and/or total daily dose of the compound of Formula (II) that can be administered while increasing efficacy and safeguarding tolerability and safety; a method of reducing an adverse event associated with treatment by the compound of Formula (II), wherein the subject is at risk of experiencing the adverse event as a result of being treated with the compound of Formula (II).

In one embodiment, the compound of Formula (I) is selected from the group consisting of clonidine, lofexidine, guanfacine, dexmedetomidine, guanabenz, tizanidine, brimonidine, xylazine and xylometazoline, an enantiomer thereof, a metabolite thereof, a derivative thereof, a prodrug thereof, a pharmaceutically acceptable salt thereof, or an acid addition salt or a combination thereof.

In another embodiment, the compound of Formula (II) is selected from the group consisting of N-(4-((6-(4-(trifluoromethyl)phenyl)pyrimidin-4-yl)oxy)benzo[d]thiazol-2-yl)acetamide, 5′-chloro-1′-(3-fluorobenzyl)-7′-methylspiro[imidazolidine-4,3′-indoline]-2,2′,5-trione, (R)-1-(5-(tert-butyl)-2,3-dihydro-1H-inden-1-yl)-3-(1H-indazol-4-yl)urea, (S)-3-(hydroxymethyl)-4-(5-methylpyridin-2-yl)-N-(4-(2,2,2-trifluoroethoxy)phenyl)-3,4-dihydro-2H-benzo[b][1,4]oxazine-8-carboxamide, (R)-1-(6-fluorospiro[chromane-2,1′-cyclobutan]-4-yl)-3-(isoquinolin-5-yl)urea, and N-(4-(trifluoromethyl)phenyl)-7-(3-(trifluoromethyl)pyridin-2-yl)quinazolin-4-amine; or an enantiomer thereof, a metabolite thereof, a derivative thereof, a prodrug thereof, a pharmaceutically acceptable salt thereof, or an acid addition salt or a combination thereof.

One aspect of the present disclosure is a method of treating pain comprising administering a TRPV1 antagonist and an alpha-2 adrenoreceptor agonist to a patient in need thereof. In some embodiments, the TRPV1 antagonist is a compound of Formula (II). In further embodiments, the alpha-2 adrenoreceptor agonist is a compound of Formula (I). In still further embodiments, the TRPV1 antagonist is a compound of Formula (II) and the alpha-2 adrenoreceptor agonist is a compound of Formula (I).

In other embodiments, the TRPV1 antagonist is selected from the group consisting of N-(4-((6-(4-(trifluoromethyl)phenyl)pyrimidin-4-yl)oxy)benzo[d]thiazol-2-yl)acetamide, 5′-chloro-1′-(3-fluorobenzyl)-7′-methylspiro[imidazolidine-4,3′-indoline]-2,2′,5-trione, (R)-1-(5-(tert-butyl)-2,3-dihydro-1H-inden-1-yl)-3-(1H-indazol-4-yl)urea, (S)-3-(hydroxymethyl)-4-(5-methylpyridin-2-yl)-N-(4-(2,2,2-trifluoroethoxy)phenyl)-3,4-dihydro-2H-benzo[b][1,4]oxazine-8-carboxamide, (R)-1-(6-fluorospiro[chromane-2,1′-cyclobutan]-4-yl)-3-(isoquinolin-5-yl)urea, and N-(4-(trifluoromethyl)phenyl)-7-(3-(trifluoromethyl)pyridin-2-yl)quinazolin-4-amine; or a pharmaceutically acceptable salt thereof, or an acid addition salt or a combination thereof.

The TRPV1 antagonist and an alpha-2 agonist may be formulated as a single pharmaceutical composition.

In embodiments where the alpha-2 agonist and the TRPV1 antagonists are administered, the alpha-2 agonist may be administered up to 3 hours before, simultaneously, or up to 3 hours after administration of TRPV1 antagonist.

The present disclosure also describes a method of treating pain, comprising administering the pharmaceutical composition of comprising an alpha-2 agonist and a TRPV1 antagonist to a patient in need thereof.

### BRIEF DESCRIPTION OF FIGURES

FIG. 1 depicts the mechanisms of interaction between control of the cardiovascular system and nociception (Sacco, M. et al., J. Clin. Hypertension, 2013, 15, 600).

FIG. 2 depicts the effect of ABT-102 on core body temperature. In the conscious, telemetry-instrumented rat, ABT-102 produced a dose-dependent and self-limiting increase in body temperature (up to 1.0° C. above vehicle) (n=7-9/group). Open circle: vehicle group; filled circle: ABT-102 10 mg/kg, p.o.; grey circle: ABT-102 3 mg/kg, p.o.

FIG. 3 depicts the effect of dexmedetomidine (Dex) on the core body temperature in the conscious, telemetry-instrumented rats (n=6-8). Open circles: vehicle group; filled circles: Dex at a dose of 0.003 mg/kg where drug was administered at time zero.

FIG. 4 depicts the effect of dexmedetomidine (Dex) on ABT-102-induced increase in the core body temperature in the conscious, telemetry-instrumented rats (n=6-8). Open circles: vehicle+vehicle group; filled circles: ABT-102 5 mg/kg, p.o. in combination with

FIG. 5 depicts the effect of dexmedetomidine (Dex, i.p.) or its vehicle in combination with ABT-102 (p.o.) or its vehicle on mean arterial blood pressure (MAP) in the conscious, telemetry-instrumented rats. Panel A: male rats (n=8/group). Panel B: female rats (n=6-8/group). Drugs were co-administered at time point ‘0’.

FIG. 6 depicts the effect of dexmedetomidine (Dex, i.p.) or its vehicle in combination with ABT-102 (p.o.) or its vehicle on tactile allodynia thresholds in male and female rats with experimental diabetes (n=6). Data are presented as geometric means. * p<0.05 (Dunn's test).

### DETAILED DESCRIPTION OF THE INVENTION

As used herein, “pain” refers to acute pain, chronic pain syndromes (including somatogenic, neurogenic, and/or psychogenic), inflammatory pain, musculoskeletal pain, neuropathic pain, cancer pain, post-operative pain, and pain associated with neuropathy and other diseases.

Somatogenic pain can be muscular or skeletal. For example, osteoarthritis, lumbosacral back pain, posttraumatic, spinal and peripheral nervous system injury, phantom pains due to amputations and myofascial pain are commonly observed somatogenic pain conditions. Maladies of the viscera such as chronic pancreatitis, ulcers, and irritable bowel disease give rise to somatogenic pain in large numbers of people. Ischemic events frequently cause somatogenic pain as in arteriosclerosis obliterans, stroke, heart attack, and angina pectoris. Cancer is also the cause of significant somatogenic pain in our society. Neurogenic pain can be due to posttraumatic and postoperative neuralgia. Neurogenic pain also can be related to degenerative neuropathies due to diabetes and can be secondary to a variety of toxic insults. Neurogenic pain can also be due to nerve entrapment, irritation or disruption, facial neuralgia, perineal neuralgia, post-amputation phantom pain, thalamic, causalgia, and reflex sympathetic dystrophy. Psychogenic pain on the other hand, is not amenable to corrective physical treatments or to pharmacological treatments that either alleviate some attribute of a pathophysiologic process. Psychogenic pain is treated instead with psychiatric interventions such as counseling and psychopharmaceuticals such as antidepressants. Chronic pain is of a protracted duration with little or no incremental improvement, usually having a duration greater than 6 months.

As used in the specification and appended claims, the terms “a,” “an,” and “the” include both singular and plural referents, unless the context clearly dictates otherwise.

Before the various embodiments are described, it is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described. All patents, publications and websites referred to herein are expressly incorporated by reference.

Various embodiments of the disclosure relate to treatment of pain, CP syndromes, inflammatory pain, cancer pain and pain associated with neuropathy and other diseases, disease conditions and co-morbidities such as those characterized by arterial hypertension, with novel compositions, combinations, therapeutic formulations, therapies, kits thereof.

In various embodiments, the invention is a composition comprising a one or more agents, each having a unique Therapeutic Mode of Action (TMA), wherein the agent is TRPV1 receptor antagonist, and/or α2-adrenoceptor agonist.

The present disclosure relates to a method of controlling pain in mammals with a composition comprising TRPV1 receptor antagonist, and/or α2-adrenoceptor agonist.

One aspect of the current disclosure relates to a method of treating pain by a composition comprising TRPV1 antagonist in combination with α2-adrenoceptor agonist, which reduces the risk of TRPV1 antagonist-induced hyperthermia.

One aspect of the current disclosure relates to a method of treating pain by a composition comprising TRPV1 antagonist in combination with α2-adrenoceptor agonist, which reduces the risk of α2-adrenoceptor agonist-induced hypotension.

In conjunction with any of the above or below embodiments, α2-adrenoceptor agonist is administered up to 3 hours before, simultaneously or up to 3 hours after administration of TRPV1 antagonist.

In conjunction with any of the above or below embodiments, α2-adrenoceptor agonist is selected from clonidine, dexmedetomidine, tizanidine, guanfacine, lofexidine, guanabenz, xylazine and xylometazoline

In conjunction with any of the above or below embodiments, the TRPV1 antagonist is selected from: N-(4-((6-(4-(trifluoromethyl)phenyl)pyrimidin-4-yl)oxy)benzo[d]thiazol-2-yl)acetamide, 5′-chloro-1′-(3-fluorobenzyl)-7′-methylspiro[imidazolidine-4,3′-indoline]-2,2′,5-trione, (R)-1-(5-(tert-butyl)-2,3-dihydro-1H-inden-1-yl)-3-(1H-indazol-4-yl)urea, (S)-3-(hydroxymethyl)-4-(5-methylpyridin-2-yl)-N-(4-(2,2,2-trifluoroethoxy)phenyl)-3,4-dihydro-2H-benzo[b] [1,4]oxazine-8-carboxamide, (R)-1-(6-fluorospiro[chromane-2,1′-cyclobutan]-4-yl)-3-(isoquinolin-5-yl)urea, and N-(4-(trifluoromethyl)phenyl)-7-(3-(trifluoromethyl)pyridin-2-yl)quinazolin-4-amine.

An embodiment of the invention is a composition comprising a compound of Formula (I)

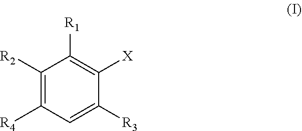

or a pharmaceutically acceptable salt or prodrug thereof, wherein

### R₁ is H, Me, or Cl;

### R₂ is H or Me; or

R₁ and R₂ together form

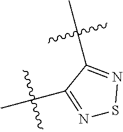 [SMILES: *c1nsnc1*]

### R₃ is H, Me, or Cl;

R₄ is H or tert-Bu;
X is selected from the group consisting of

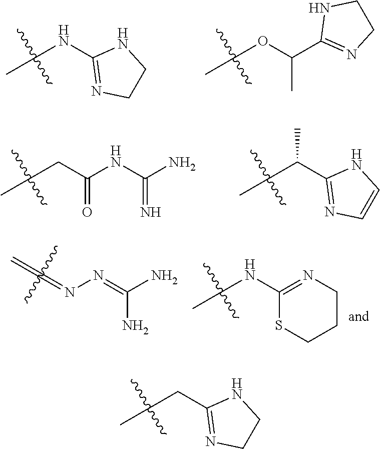

An embodiment of the invention is a composition comprising a compound of Formula (II)

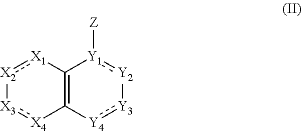

or a pharmaceutically acceptable salt or prodrug thereof, wherein [symbol]
is absent or single bond

### X₁ is CH, CMe, N or O;

X₂ is CH, CH₂, CNHAc or N;
X₃ is CR₁, (S)—CHCH₂OH, N, NH, or S;
X₄ is bond, CH or NR₂;

### R₁ is

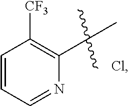 [SMILES: *c1ncccc1C(F)(F)F]

### R₂ is

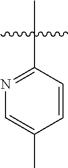 [SMILES: *c1ccc(C)cn1]

### Y₁ is C or N;

### Y₂ is CH, C═O or N;

Y₃ is CH or C-G, where G is a spiro-ring

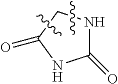

Y₄ is bond, CH or N;

### Z is

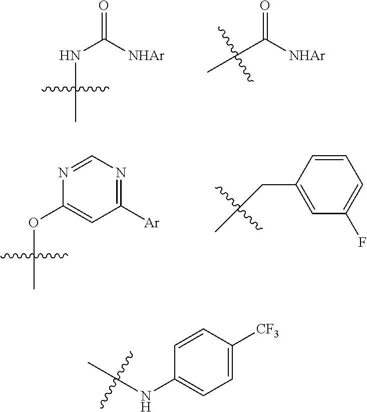

### Ar is

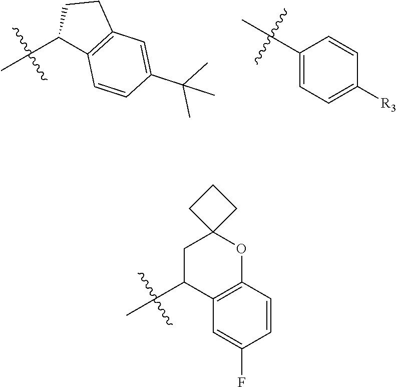

R₃ is CF₃ and OCH₂CF₃.

Some embodiments include a method of treating a disease or disorder in a subject in need thereof comprising an effective amount of: 1) a composition comprising a compound of Formula (I), as defined above; enantiomers thereof, metabolites thereof, derivatives thereof, and/or prodrugs thereof, pharmaceutically acceptable salts thereof, or a combination thereof; or 2) a compound of Formula (II), as defined above, enantiomers, metabolites, derivatives, prodrugs, salts, diastereomers, pharmaceutically acceptable salts, or a combination thereof; or 3) a combination of 1 and 2.

In an embodiment, the method is a method of decreasing the number of doses and/or total daily dose of the compound of Formula (II) that can be administered while increasing efficacy and safeguarding tolerability and safety; a method of reducing an adverse event associated with treatment by the compound of Formula (II), wherein the subject is at risk of experiencing the adverse event as a result being treated with the compound of Formula (II).

In an embodiment, the method is a method of decreasing the number of doses and/or total daily dose of the compound of Formula (I) that can be administered while increasing efficacy and safeguarding tolerability and safety; a method of reducing an adverse event associated with treatment by the compound of Formula (I), wherein the subject is at risk of experiencing the adverse event as a result being treated with the compound of Formula (I).

Another embodiment is a composition comprising a compound of Formula (I), and a compound of Formula (II), wherein the compounds of Formula (I) and Formula (II) thereof are independently an acid addition salt: hydrogen acetate, hydrogen acetyl salicylate, hydrogen adipate, hydrogen aspartate, hydrogen butyrate, hydrogen caprate, hydrogen caproate, hydrogen caprylate, hydrogen enanthate, hydrogen formate, hydrogen fumarate, hydrogen glutamate, hydrogen glutarate, hydrogen isophthalate, hydrogen maleate, hydrogen malonate, hydrogen methionate, hydrogen oxalate, hydrogen pelargonate, hydrogen pimelate, hydrogen propionate, hydrogen phthalate, hydrogen salicylate, hydrogen sebacate, hydrogen succinate, hydrogen terephthalate, hydrogen tyrosinate, hydrogen tryptophanate, hydrogen valerate, hydrogen N-acyl-aspartate, hydrogen N-acyl-glutamate, hydrogen N-acyl-tyrosinate, hydrogen N-acyl-tryptophanate, hydrogen N-acyl-methionate, hydrogen citrate, hydrogen galactonate, hydrogen glucaric acid (saccharic acid), hydrogen mannonate, hydrogen mucate, hydrogen rhamnonate, and hydrogen tartrate.

An embodiment of the invention is an addition salt of Formula (I), wherein with organic acid such as aspartic acid, benzenesulfonic acid, besylic acid, benzoic acid, bicarbonic acid, tartaric acid, bromide, camphor sulfonic acid, camsylic acid, chloride, citric acid, decanoic acid, edetate, lauryl sulfonic acid, estolic acid, ethanesulfonic acid, esylic acid, fumaric acid, gluceptic acid, gluconic acid, glutamic acid, glycolic acid, glycoloylarsanilic acid, hexanoic acid, hexylresorcinol, hydroxynaphthoic acid, isethionic acid, iodide, lactic acid, galactopyranosyl-d-gluconic acid, lactobionic acid, malic acid, maleic acid, mandelic acid, methanesulfonic acid, methylbromide, methylcitric acid, methylsulfonic acid, mucic acid, napsylic acid, nitric acid, octanoic acid, oleic acid, pamoic acid, 4,4′-methylenebis(3-hydroxy-2-naphthonic acid, pantothenic acid, phosphoric acid, polygalacturonic acid, propionic acid, salicylic acid, stearic acid, succinic acid, sulfuric acid, tartaric acid, teoclic acid, 8-chloro-1,3-dimethyl-7h-purine-2,6-dione, tosylic acid, malic acid, methionic acid, phthallic acid, malonic acid, tyrosine, tryptophan, maleic acid, fumaric acid, succinic acid, glutaric acid, adipic acid, pimelic acid, sebacic acid, formic acid, acetic acid, propionic acid, butyric acid, valeric acid, caproic acid, enanthic acid, caprylic acid, pelargonic acid, capric acid, oxalic acid, isophthallic acid, terephthallic acid, salicylic acid, difluorosuccinicacid, trifluorosuccinic acid, tetrafluorosuccinic acid, difluoroglutaric acid, difluoroacetic acid, trifluoroacetic acid; and dextromethorphan; or a combination thereof.

Another embodiment is a composition comprising a compound of Formula (I), and at least one compound selected from Formula (II):

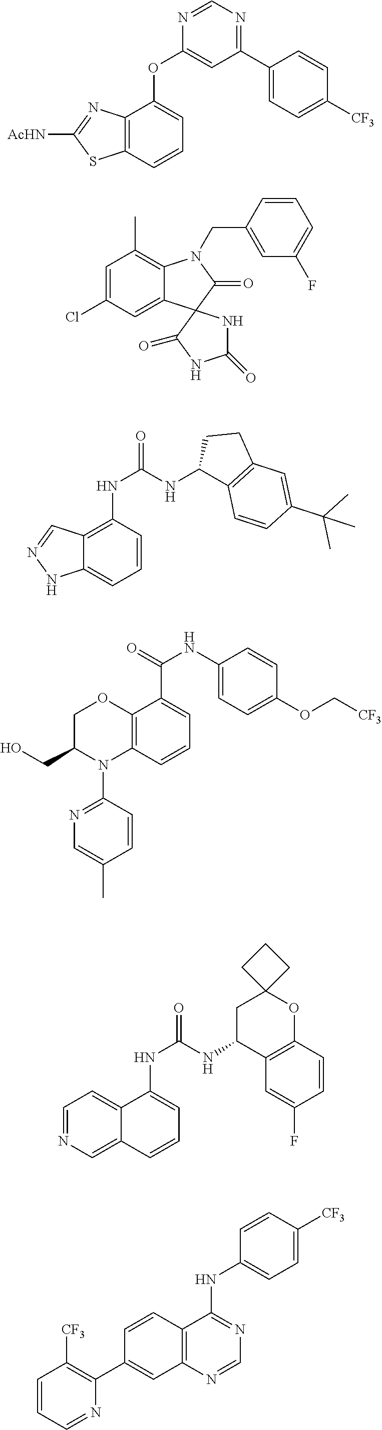 [SMILES: CC(=O)Nc1nc2c(Oc3cc(-c4ccc(C(F)(F)F)cc4)ncn3)cccc2s1 ; Cc1cc(Cl)cc2c1N(Cc1cccc(F)c1)C(=O)C21NC(=O)NC1=O ; CC(C)(C)c1ccc2c(c1)CC[C@H]2NC(=O)Nc1cccc2[nH]ncc12 ; Cc1ccc(N2c3cccc(C(=O)Nc4ccc(OCC(F)(F)F)cc4)c3OC[C@@H]2CO)nc1 ; O=C(Nc1cccc2cnccc12)N[C@@H]1CC2(CCC2)Oc2ccc(F)cc21 ; FC(F)(F)c1ccc(Nc2ncnc3cc(-c4ncccc4C(F)(F)F)ccc23)cc1]

Another embodiment is a composition comprising a compound of Formula (II), and at least one compound selected from Formula (I):

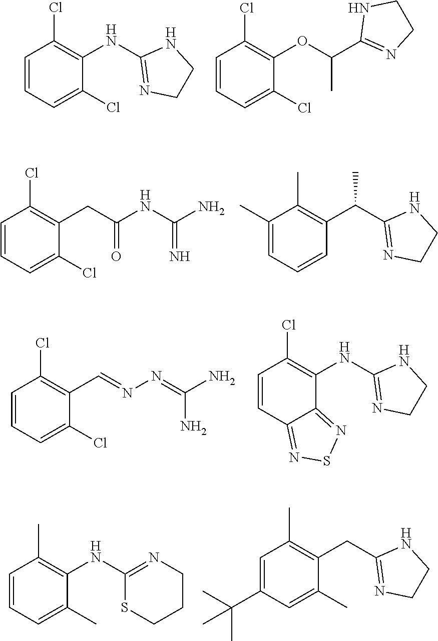

Another embodiment is a composition comprising clonidine (or a pharmaceutically acceptable salt thereof) and a compound selected from the group consisting of N-(4-((6-(4-(trifluoromethyl)phenyl)pyrimidin-4-yl)oxy)benzo[d]thiazol-2-yl)acetamide, 5′-chloro-1′-(3-fluorobenzyl)-7′-methylspiro[imidazolidine-4,3′-indoline]-2,2′,5-trione, (R)-1-(5-(tert-butyl)-2,3-dihydro-1H-inden-1-yl)-3-(1H-indazol-4-yl)urea, (S)-3-(hydroxymethyl)-4-(5-methylpyridin-2-yl)-N-(4-(2,2,2-trifluoroethoxy)phenyl)-3,4-dihydro-2H-benzo[b][1,4]oxazine-8-carboxamide, (R)-1-(6-fluorospiro[chromane-2,1′-cyclobutan]-4-yl)-3-(isoquinolin-5-yl)urea, and N-(4-(trifluoromethyl)phenyl)-7-(3-(trifluoromethyl)pyridin-2-yl)quinazolin-4-amine; or a pharmaceutically acceptable salt thereof.

Another embodiment is a composition comprising lofexidine (or a pharmaceutically acceptable salt thereof) and a compound selected from the group consisting of N-(4-((6-(4-(trifluoromethyl)phenyl)pyrimidin-4-yl)oxy)benzo[d]thiazol-2-yl)acetamide, 5′-chloro-1′-(3-fluorobenzyl)-7′-methylspiro[imidazolidine-4,3′-indoline]-2,2′,5-trione, (R)-1-(5-(tert-butyl)-2,3-dihydro-1H-inden-1-yl)-3-(1H-indazol-4-yl)urea, (S)-3-(hydroxymethyl)-4-(5-methylpyridin-2-yl)-N-(4-(2,2,2-trifluoroethoxy)phenyl)-3,4-dihydro-2H-benzo[b][1,4]oxazine-8-carboxamide, (R)-1-(6-fluorospiro[chromane-2,1′-cyclobutan]-4-yl)-3-(isoquinolin-5-yl)urea, and N-(4-(trifluoromethyl)phenyl)-7-(3-(trifluoromethyl)pyridin-2-yl)quinazolin-4-amine; or a pharmaceutically acceptable salt thereof.

Another embodiment is a composition comprising guanfacine (or a pharmaceutically acceptable salt thereof) and a compound selected from the group consisting of N-(4-((6-(4-(trifluoromethyl)phenyl)pyrimidin-4-yl)oxy)benzo[d]thiazol-2-yl)acetamide, 5′-chloro-1′-(3-fluorobenzyl)-7′-methylspiro[imidazolidine-4,3′-indoline]-2,2′,5-trione, (R)-1-(5-(tert-butyl)-2,3-dihydro-1H-inden-1-yl)-3-(1H-indazol-4-yl)urea, (S)-3-(hydroxymethyl)-4-(5-methylpyridin-2-yl)-N-(4-(2,2,2-trifluoroethoxy)phenyl)-3,4-dihydro-2H-benzo[b][1,4]oxazine-8-carboxamide, (R)-1-(6-fluorospiro[chromane-2,1′-cyclobutan]-4-yl)-3-(isoquinolin-5-yl)urea, and N-(4-(trifluoromethyl)phenyl)-7-(3-(trifluoromethyl)pyridin-2-yl)quinazolin-4-amine; or a pharmaceutically acceptable salt thereof.

Another embodiment is a composition comprising dexmedetomidine (or a pharmaceutically acceptable salt thereof) and a compound selected from the group consisting of N-(4-((6-(4-(trifluoromethyl)phenyl)pyrimidin-4-yl)oxy)benzo[d]thiazol-2-yl)acetamide, 5′-chloro-1′-(3-fluorobenzyl)-7′-methylspiro[imidazolidine-4,3′-indoline]-2,2′,5-trione, (R)-1-(5-(tert-butyl)-2,3-dihydro-1H-inden-1-yl)-3-(1H-indazol-4-yl)urea, (S)-3-(hydroxymethyl)-4-(5-methylpyridin-2-yl)-N-(4-(2,2,2-trifluoroethoxy)phenyl)-3,4-dihydro-2H-benzo[b][1,4]oxazine-8-carboxamide, (R)-1-(6-fluorospiro[chromane-2,1′-cyclobutan]-4-yl)-3-(isoquinolin-5-yl)urea, and N-(4-(trifluoromethyl)phenyl)-7-(3-(trifluoromethyl)pyridin-2-yl)quinazolin-4-amine; or a pharmaceutically acceptable salt thereof.

Another embodiment is a composition comprising guanabenz (or a pharmaceutically acceptable salt thereof) and a compound selected from the group consisting of N-(4-((6-(4-(trifluoromethyl)phenyl)pyrimidin-4-yl)oxy)benzo[d]thiazol-2-yl)acetamide, 5′-chloro-1′-(3-fluorobenzyl)-7′-methylspiro[imidazolidine-4,3′-indoline]-2,2′,5-trione, (R)-1-(5-(tert-butyl)-2,3-dihydro-1H-inden-1-yl)-3-(1H-indazol-4-yl)urea, (S)-3-(hydroxymethyl)-4-(5-methylpyridin-2-yl)-N-(4-(2,2,2-trifluoroethoxy)phenyl)-3,4-dihydro-2H-benzo[b][1,4]oxazine-8-carboxamide, (R)-1-(6-fluorospiro[chromane-2,1′-cyclobutan]-4-yl)-3-(isoquinolin-5-yl)urea, and N-(4-(trifluoromethyl)phenyl)-7-(3-(trifluoromethyl)pyridin-2-yl)quinazolin-4-amine; or a pharmaceutically acceptable salt thereof.

Another embodiment is a composition comprising tizanidine (or a pharmaceutically acceptable salt thereof) and a compound selected from the group consisting of N-(4-((6-(4-(trifluoromethyl)phenyl)pyrimidin-4-yl)oxy)benzo[d]thiazol-2-yl)acetamide, 5′-chloro-1′-(3-fluorobenzyl)-7′-methylspiro[imidazolidine-4,3′-indoline]-2,2′,5-trione, (R)-1-(5-(tert-butyl)-2,3-dihydro-1H-inden-1-yl)-3-(1H-indazol-4-yl)urea, (S)-3-(hydroxymethyl)-4-(5-methylpyridin-2-yl)-N-(4-(2,2,2-trifluoroethoxy)phenyl)-3,4-dihydro-2H-benzo[b][1,4]oxazine-8-carboxamide, (R)-1-(6-fluorospiro[chromane-2,1′-cyclobutan]-4-yl)-3-(isoquinolin-5-yl)urea, and N-(4-(trifluoromethyl)phenyl)-7-(3-(trifluoromethyl)pyridin-2-yl)quinazolin-4-amine; or a pharmaceutically acceptable salt thereof.

Another embodiment is a composition comprising brimonidine (or a pharmaceutically acceptable salt thereof) and a compound selected from the group consisting of N-(4-((6-(4-(trifluoromethyl)phenyl)pyrimidin-4-yl)oxy)benzo[d]thiazol-2-yl)acetamide, 5′-chloro-1′-(3-fluorobenzyl)-7′-methylspiro[imidazolidine-4,3′-indoline]-2,2′,5-trione, (R)-1-(5-(tert-butyl)-2,3-dihydro-1H-inden-1-yl)-3-(1H-indazol-4-yl)urea, (S)-3-(hydroxymethyl)-4-(5-methylpyridin-2-yl)-N-(4-(2,2,2-trifluoroethoxy)phenyl)-3,4-dihydro-2H-benzo[b][1,4]oxazine-8-carboxamide, (R)-1-(6-fluorospiro[chromane-2,1′-cyclobutan]-4-yl)-3-(isoquinolin-5-yl)urea, and N-(4-(trifluoromethyl)phenyl)-7-(3-(trifluoromethyl)pyridin-2-yl)quinazolin-4-amine; or a pharmaceutically acceptable salt thereof.

Another embodiment is a composition comprising xylazine (or a pharmaceutically acceptable salt thereof) and a compound selected from the group consisting of N-(4-((6-(4-(trifluoromethyl)phenyl)pyrimidin-4-yl)oxy)benzo[d]thiazol-2-yl)acetamide, 5′-chloro-1′-(3-fluorobenzyl)-7′-methylspiro[imidazolidine-4,3′-indoline]-2,2′,5-trione, (R)-1-(5-(tert-butyl)-2,3-dihydro-1H-inden-1-yl)-3-(1H-indazol-4-yl)urea, (S)-3-(hydroxymethyl)-4-(5-methylpyridin-2-yl)-N-(4-(2,2,2-trifluoroethoxy)phenyl)-3,4-dihydro-2H-benzo[b][1,4]oxazine-8-carboxamide, (R)-1-(6-fluorospiro[chromane-2,1′-cyclobutan]-4-yl)-3-(isoquinolin-5-yl)urea, and N-(4-(trifluoromethyl)phenyl)-7-(3-(trifluoromethyl)pyridin-2-yl)quinazolin-4-amine; or a pharmaceutically acceptable salt thereof.

Another embodiment is a composition comprising xylometazoline (or a pharmaceutically acceptable salt thereof) and a compound selected from the group consisting of N-(4-((6-(4-(trifluoromethyl)phenyl)pyrimidin-4-yl)oxy)benzo[d]thiazol-2-yl)acetamide, 5′-chloro-1′-(3-fluorobenzyl)-7′-methylspiro[imidazolidine-4,3′-indoline]-2,2′,5-trione, (R)-1-(5-(tert-butyl)-2,3-dihydro-1H-inden-1-yl)-3-(1H-indazol-4-yl)urea, (S)-3-(hydroxymethyl)-4-(5-methylpyridin-2-yl)-N-(4-(2,2,2-trifluoroethoxy)phenyl)-3,4-dihydro-2H-benzo[b][1,4]oxazine-8-carboxamide, (R)-1-(6-fluorospiro[chromane-2,1′-cyclobutan]-4-yl)-3-(isoquinolin-5-yl)urea, and N-(4-(trifluoromethyl)phenyl)-7-(3-(trifluoromethyl)pyridin-2-yl)quinazolin-4-amine; or a pharmaceutically acceptable salt thereof.

Another embodiment is a composition comprising N-(4-((6-(4-(trifluoromethyl)phenyl)pyrimidin-4-yl)oxy)benzo[d]thiazol-2-yl)acetamide (or a pharmaceutically acceptable salt thereof) and a compound selected from the group consisting of clonidine, lofexidine, guanfacine, dexmedetomidine, guanabenz, tizanidine, brimonidine, xylazine and xylometazoline; or a pharmaceutically acceptable salt thereof.

Another embodiment is a composition comprising 5′-chloro-1′-(3-fluorobenzyl)-7′-methylspiro[imidazolidine-4,3′-indoline]-2,2′,5-trione (or a pharmaceutically acceptable salt thereof) and a compound selected from the group consisting of clonidine, lofexidine, guanfacine, dexmedetomidine, guanabenz, tizanidine, brimonidine, xylazine and xylometazoline; or a pharmaceutically acceptable salt thereof.

Another embodiment is a composition comprising (R)-1-(5-(tert-butyl)-2,3-dihydro-1H-inden-1-yl)-3-(1H-indazol-4-yl)urea (or a pharmaceutically acceptable salt thereof) and a compound selected from the group consisting of clonidine, lofexidine, guanfacine, dexmedetomidine, guanabenz, tizanidine, brimonidine, xylazine and xylometazoline; or a pharmaceutically acceptable salt thereof.

Another embodiment is a composition comprising (S)-3-(hydroxymethyl)-4-(5-methylpyridin-2-yl)-N-(4-(2,2,2-trifluoroethoxy)phenyl)-3,4-dihydro-2H-benzo[b][1,4]oxazine-8-carboxamide (or a pharmaceutically acceptable salt thereof) and a compound selected from the group consisting of clonidine, lofexidine, guanfacine, dexmedetomidine, guanabenz, tizanidine, brimonidine, xylazine and xylometazoline; or a pharmaceutically acceptable salt thereof.

Another embodiment is a composition comprising (R)-1-(6-fluorospiro[chromane-2,1′-cyclobutan]-4-yl)-3-(isoquinolin-5-yl)urea (or a pharmaceutically acceptable salt thereof) and a compound selected from the group consisting of clonidine, lofexidine, guanfacine, dexmedetomidine, guanabenz, tizanidine, brimonidine, xylazine and xylometazoline; or a pharmaceutically acceptable salt thereof.

Another embodiment is a composition comprising N-(4-(trifluoromethyl)phenyl)-7-(3-(trifluoromethyl)pyridin-2-yl)quinazolin-4-amine (or a pharmaceutically acceptable salt thereof) and a compound selected from the group consisting of clonidine, lofexidine, guanfacine, dexmedetomidine, guanabenz, tizanidine, brimonidine, xylazine and xylometazoline; or a pharmaceutically acceptable salt thereof.

Another embodiment is a composition comprising clonidine, or a pharmaceutically acceptable salt thereof, and N-(4-((6-(4-(trifluoromethyl)phenyl)pyrimidin-4-yl)oxy)benzo[d]thiazol-2-yl)acetamide, or a pharmaceutically acceptable salt thereof.

Another embodiment is a composition comprising clonidine, or a pharmaceutically acceptable salt thereof, and 5′-chloro-1′-(3-fluorobenzyl)-7′-methylspiro[imidazolidine-4,3′-indoline]-2,2′,5-trione, or a pharmaceutically acceptable salt thereof.

Another embodiment is a composition comprising clonidine, or a pharmaceutically acceptable salt thereof, and (R)-1-(5-(tert-butyl)-2,3-dihydro-1H-inden-1-yl)-3-(1H-indazol-4-yl)urea, or a pharmaceutically acceptable salt thereof.

Another embodiment is a composition comprising clonidine, or a pharmaceutically acceptable salt thereof, and (S)-3-(hydroxymethyl)-4-(5-methylpyridin-2-yl)-N-(4-(2,2,2-trifluoroethoxy)phenyl)-3,4-dihydro-2H-benzo[b][1,4]oxazine-8-carboxamide, or a pharmaceutically acceptable salt thereof.

Another embodiment is a composition comprising clonidine, or a pharmaceutically acceptable salt thereof, and (R)-1-(6-fluorospiro[chromane-2,1′-cyclobutan]-4-yl)-3-(isoquinolin-5-yl)urea or a pharmaceutically acceptable salt thereof.

Another embodiment is a composition comprising clonidine, or a pharmaceutically acceptable salt thereof, and N-(4-(trifluoromethyl)phenyl)-7-(3-(trifluoromethyl)pyridin-2-yl)quinazolin-4-amine, or a pharmaceutically acceptable salt thereof.

Another embodiment is a composition comprising lofexidine, or a pharmaceutically acceptable salt thereof, and N-(4-((6-(4-(trifluoromethyl)phenyl)pyrimidin-4-yl)oxy)benzo[d]thiazol-2-yl)acetamide, or a pharmaceutically acceptable salt thereof.

Another embodiment is a composition comprising lofexidine, or a pharmaceutically acceptable salt thereof, and 5′-chloro-1′-(3-fluorobenzyl)-7′-methylspiro[imidazolidine-4,3′-indoline]-2,2′,5-trione, or a pharmaceutically acceptable salt thereof.

Another embodiment is a composition comprising lofexidine, or a pharmaceutically acceptable salt thereof, and (R)-1-(5-(tert-butyl)-2,3-dihydro-1H-inden-1-yl)-3-(1H-indazol-4-yl)urea, or a pharmaceutically acceptable salt thereof.

Another embodiment is a composition comprising lofexidine, or a pharmaceutically acceptable salt thereof, and (S)-3-(hydroxymethyl)-4-(5-methylpyridin-2-yl)-N-(4-(2,2,2-trifluoroethoxy)phenyl)-3,4-dihydro-2H-benzo[b][1,4]oxazine-8-carboxamide, or a pharmaceutically acceptable salt thereof.

Another embodiment is a composition comprising lofexidine, or a pharmaceutically acceptable salt thereof, and (R)-1-(6-fluorospiro[chromane-2,1′-cyclobutan]-4-yl)-3-(isoquinolin-5-yl)urea, or a pharmaceutically acceptable salt thereof.

Another embodiment is a composition comprising lofexidine, or a pharmaceutically acceptable salt thereof, and N-(4-(trifluoromethyl)phenyl)-7-(3-(trifluoromethyl)pyridin-2-yl)quinazolin-4-amine, or a pharmaceutically acceptable salt thereof.

Another embodiment is a composition comprising guanfacine, or a pharmaceutically acceptable salt thereof, and N-(4-((6-(4-(trifluoromethyl)phenyl)pyrimidin-4-yl)oxy)benzo[d]thiazol-2-yl)acetamide, or a pharmaceutically acceptable salt thereof.

Another embodiment is a composition comprising guanfacine, or a pharmaceutically acceptable salt thereof, and 5′-chloro-1′-(3-fluorobenzyl)-7′-methylspiro[imidazolidine-4,3′-indoline]-2,2′,5-trione, or a pharmaceutically acceptable salt thereof.

Another embodiment is a composition comprising guanfacine, or a pharmaceutically acceptable salt thereof, and (R)-1-(5-(tert-butyl)-2,3-dihydro-1H-inden-1-yl)-3-(1H-indazol-4-yl)urea, or a pharmaceutically acceptable salt thereof.

Another embodiment is a composition comprising guanfacine, or a pharmaceutically acceptable salt thereof, and (S)-3-(hydroxymethyl)-4-(5-methylpyridin-2-yl)-N-(4-(2,2,2-trifluoroethoxy)phenyl)-3,4-dihydro-2H-benzo[b][1,4]oxazine-8-carboxamide, or a pharmaceutically acceptable salt thereof.

Another embodiment is a composition comprising guanfacine, or a pharmaceutically acceptable salt thereof, and (R)-1-(6-fluorospiro[chromane-2,1′-cyclobutan]-4-yl)-3-(isoquinolin-5-yl)urea, or a pharmaceutically acceptable salt thereof.

Another embodiment is a composition comprising guanfacine, or a pharmaceutically acceptable salt thereof, and N-(4-(trifluoromethyl)phenyl)-7-(3-(trifluoromethyl)pyridin-2-yl)quinazolin-4-amine, or a pharmaceutically acceptable salt thereof.

Another embodiment is a composition comprising dexmedetomidine, or a pharmaceutically acceptable salt thereof, and N-(4-((6-(4-(trifluoromethyl)phenyl)pyrimidin-4-yl)oxy)benzo[d]thiazol-2-yl)acetamide, or a pharmaceutically acceptable salt thereof.

Another embodiment is a composition comprising dexmedetomidine, or a pharmaceutically acceptable salt thereof, and 5′-chloro-1′-(3-fluorobenzyl)-7′-methylspiro[imidazolidine-4,3′-indoline]-2,2′,5-trione, or a pharmaceutically acceptable salt thereof.

Another embodiment is a composition comprising dexmedetomidine, or a pharmaceutically acceptable salt thereof, and (R)-1-(5-(tert-butyl)-2,3-dihydro-1H-inden-1-yl)-3-(1H-indazol-4-yl)urea, or a pharmaceutically acceptable salt thereof.

Another embodiment is a composition comprising dexmedetomidine, or a pharmaceutically acceptable salt thereof, and (S)-3-(hydroxymethyl)-4-(5-methylpyridin-2-yl)-N-(4-(2,2,2-trifluoroethoxy)phenyl)-3,4-dihydro-2H-benzo[b][1,4]oxazine-8-carboxamide, or a pharmaceutically acceptable salt thereof.

Another embodiment is a composition comprising dexmedetomidine, or a pharmaceutically acceptable salt thereof, and (R)-1-(6-fluorospiro[chromane-2,1′-cyclobutan]-4-yl)-3-(isoquinolin-5-yl)urea, or a pharmaceutically acceptable salt thereof.

Another embodiment is a composition comprising dexmedetomidine, or a pharmaceutically acceptable salt thereof, and N-(4-(trifluoromethyl)phenyl)-7-(3-(trifluoromethyl)pyridin-2-yl)quinazolin-4-amine, or a pharmaceutically acceptable salt thereof.

Another embodiment is a composition comprising guanabenz, or a pharmaceutically acceptable salt thereof, and N-(4-((6-(4-(trifluoromethyl)phenyl)pyrimidin-4-yl)oxy)benzo[d]thiazol-2-yl)acetamide, or a pharmaceutically acceptable salt thereof.

Another embodiment is a composition comprising guanabenz, or a pharmaceutically acceptable salt thereof, and 5′-chloro-1′-(3-fluorobenzyl)-7′-methylspiro[imidazolidine-4,3′-indoline]-2,2′,5-trione, or a pharmaceutically acceptable salt thereof.

Another embodiment is a composition comprising guanabenz, or a pharmaceutically acceptable salt thereof, and (R)-1-(5-(tert-butyl)-2,3-dihydro-1H-inden-1-yl)-3-(H-indazol-4-yl)urea, or a pharmaceutically acceptable salt thereof.

Another embodiment is a composition comprising guanabenz, or a pharmaceutically acceptable salt thereof, and (S)-3-(hydroxymethyl)-4-(5-methylpyridin-2-yl)-N-(4-(2,2,2-trifluoroethoxy)phenyl)-3,4-dihydro-2H-benzo[b][1,4]oxazine-8-carboxamide, or a pharmaceutically acceptable salt thereof.

Another embodiment is a composition comprising guanabenz, or a pharmaceutically acceptable salt thereof, and (R)-1-(6-fluorospiro[chromane-2,1′-cyclobutan]-4-yl)-3-(isoquinolin-5-yl)urea, or a pharmaceutically acceptable salt thereof.

Another embodiment is a composition comprising guanabenz, or a pharmaceutically acceptable salt thereof, and N-(4-(trifluoromethyl)phenyl)-7-(3-(trifluoromethyl)pyridin-2-yl)quinazolin-4-amine, or a pharmaceutically acceptable salt thereof.

Another embodiment is a composition comprising tizanidine, or a pharmaceutically acceptable salt thereof, and N-(4-((6-(4-(trifluoromethyl)phenyl)pyrimidin-4-yl)oxy)benzo[d]thiazol-2-yl)acetamide, or a pharmaceutically acceptable salt thereof.

Another embodiment is a composition comprising tizanidine, or a pharmaceutically acceptable salt thereof, and 5′-chloro-1′-(3-fluorobenzyl)-7′-methylspiro[imidazolidine-4,3′-indoline]-2,2′,5-trione, or a pharmaceutically acceptable salt thereof.

Another embodiment is a composition comprising tizanidine, or a pharmaceutically acceptable salt thereof, and (R)-1-(5-(tert-butyl)-2,3-dihydro-1H-inden-1-yl)-3-(1H-indazol-4-yl)urea, or a pharmaceutically acceptable salt thereof.

Another embodiment is a composition comprising tizanidine, or a pharmaceutically acceptable salt thereof, and (S)-3-(hydroxymethyl)-4-(5-methylpyridin-2-yl)-N-(4-(2,2,2-trifluoroethoxy)phenyl)-3,4-dihydro-2H-benzo[b][1,4]oxazine-8-carboxamide, or a pharmaceutically acceptable salt thereof.

Another embodiment is a composition comprising tizanidine, or a pharmaceutically acceptable salt thereof, and (R)-1-(6-fluorospiro[chromane-2,1′-cyclobutan]-4-yl)-3-(isoquinolin-5-yl)urea, or a pharmaceutically acceptable salt thereof.

Another embodiment is a composition comprising tizanidine, or a pharmaceutically acceptable salt thereof, and N-(4-(trifluoromethyl)phenyl)-7-(3-(trifluoromethyl)pyridin-2-yl)quinazolin-4-amine, or a pharmaceutically acceptable salt thereof.

Another embodiment is a composition comprising brimonidine, or a pharmaceutically acceptable salt thereof, and N-(4-((6-(4-(trifluoromethyl)phenyl)pyrimidin-4-yl)oxy)benzo[d]thiazol-2-yl)acetamide, or a pharmaceutically acceptable salt thereof.

Another embodiment is a composition comprising brimonidine, or a pharmaceutically acceptable salt thereof, and 5′-chloro-1′-(3-fluorobenzyl)-7′-methylspiro[imidazolidine-4,3′-indoline]-2,2′,5-trione, or a pharmaceutically acceptable salt thereof.

Another embodiment is a composition comprising brimonidine, or a pharmaceutically acceptable salt thereof, and (R)-1-(5-(tert-butyl)-2,3-dihydro-1H-inden-1-yl)-3-(H-indazol-4-yl)urea, or a pharmaceutically acceptable salt thereof.

Another embodiment is a composition comprising brimonidine, or a pharmaceutically acceptable salt thereof, and (S)-3-(hydroxymethyl)-4-(5-methylpyridin-2-yl)-N-(4-(2,2,2-trifluoroethoxy)phenyl)-3,4-dihydro-2H-benzo[b][1,4]oxazine-8-carboxamide, or a pharmaceutically acceptable salt thereof.

Another embodiment is a composition comprising brimonidine, or a pharmaceutically acceptable salt thereof, and (R)-1-(6-fluorospiro[chromane-2,1′-cyclobutan]-4-yl)-3-(isoquinolin-5-yl)urea, or a pharmaceutically acceptable salt thereof.

Another embodiment is a composition comprising brimonidine, or a pharmaceutically acceptable salt thereof, and N-(4-(trifluoromethyl)phenyl)-7-(3-(trifluoromethyl)pyridin-2-yl)quinazolin-4-amine, or a pharmaceutically acceptable salt thereof.

Another embodiment is a composition comprising xylazine, or a pharmaceutically acceptable salt thereof, and N-(4-((6-(4-(trifluoromethyl)phenyl)pyrimidin-4-yl)oxy)benzo[d]thiazol-2-yl)acetamide, or a pharmaceutically acceptable salt thereof.

Another embodiment is a composition comprising xylazine, or a pharmaceutically acceptable salt thereof, and 5′-chloro-1′-(3-fluorobenzyl)-7′-methylspiro[imidazolidine-4,3′-indoline]-2,2′,5-trione, or a pharmaceutically acceptable salt thereof.

Another embodiment is a composition comprising xylazine, or a pharmaceutically acceptable salt thereof, and (R)-1-(5-(tert-butyl)-2,3-dihydro-1H-inden-1-yl)-3-(H-indazol-4-yl)urea, or a pharmaceutically acceptable salt thereof.

Another embodiment is a composition comprising xylazine, or a pharmaceutically acceptable salt thereof, and (S)-3-(hydroxymethyl)-4-(5-methylpyridin-2-yl)-N-(4-(2,2,2-trifluoroethoxy)phenyl)-3,4-dihydro-2H-benzo[b][1,4]oxazine-8-carboxamide, or a pharmaceutically acceptable salt thereof.

Another embodiment is a composition comprising xylazine, or a pharmaceutically acceptable salt thereof, and (R)-1-(6-fluorospiro[chromane-2,1′-cyclobutan]-4-yl)-3-(isoquinolin-5-yl)urea, or a pharmaceutically acceptable salt thereof.

Another embodiment is a composition comprising xylazine, or a pharmaceutically acceptable salt thereof, and N-(4-(trifluoromethyl)phenyl)-7-(3-(trifluoromethyl)pyridin-2-yl)quinazolin-4-amine, or a pharmaceutically acceptable salt thereof.

Another embodiment is a composition comprising xylometazoline, or a pharmaceutically acceptable salt thereof, and N-(4-((6-(4-(trifluoromethyl)phenyl)pyrimidin-4-yl)oxy)benzo[d]thiazol-2-yl)acetamide, or a pharmaceutically acceptable salt thereof.

Another embodiment is a composition comprising xylometazoline, or a pharmaceutically acceptable salt thereof, and 5′-chloro-1′-(3-fluorobenzyl)-7′-methylspiro[imidazolidine-4,3′-indoline]-2,2′,5-trione, or a pharmaceutically acceptable salt thereof.

Another embodiment is a composition comprising xylometazoline, or a pharmaceutically acceptable salt thereof, and (R)-1-(5-(tert-butyl)-2,3-dihydro-1H-inden-1-yl)-3-(1H-indazol-4-yl)urea, or a pharmaceutically acceptable salt thereof.

Another embodiment is a composition comprising xylometazoline, or a pharmaceutically acceptable salt thereof, and (S)-3-(hydroxymethyl)-4-(5-methylpyridin-2-yl)-N-(4-(2,2,2-trifluoroethoxy)phenyl)-3,4-dihydro-2H-benzo[b][1,4]oxazine-8-carboxamide, or a pharmaceutically acceptable salt thereof.

Another embodiment is a composition comprising xylometazoline, or a pharmaceutically acceptable salt thereof, and (R)-1-(6-fluorospiro[chromane-2,1′-cyclobutan]-4-yl)-3-(isoquinolin-5-yl)urea, or a pharmaceutically acceptable salt thereof.

Another embodiment is a composition comprising xylometazoline, or a pharmaceutically acceptable salt thereof, and N-(4-(trifluoromethyl)phenyl)-7-(3-(trifluoromethyl)pyridin-2-yl)quinazolin-4-amine, or a pharmaceutically acceptable salt thereof.

Methods of Use

These methods may be used to treat or provide relief to, any pain including, but not limited to, chronic pain (e.g., somatogenic, neurogenic, or psychogenic pain), musculoskeletal pain, neuropathic pain, cancer-related pain, acute pain, nociceptive pain, etc. Examples of musculoskeletal pain include low back pain (i.e. lumbosacral pain), primary dysmenorrhea, and arthritic pain, such as pain associated with rheumatoid arthritis, juvenile rheumatoid arthritis, osteoarthritis, osteoarthosis, axial spondyloarthritis including ankylosing spondylitis, etc.

Examples of neuropathic pain include idiopathic and diabetic peripheral neuropathy, post-herpetic neuralgia, trigeminal neuralgia, monoradiculopathies, phantom limb pain, central pain, etc. Other causes of neuropathic pain include cancer-related pain, lumbar nerve root compression, spinal cord injury, post-stroke pain, central multiple sclerosis pain, HIV-associated neuropathy, and radio- or chemo-therapy associated neuropathy, etc.

The term “treating” or “treatment” includes the diagnosis, cure, mitigation, treatment, or prevention of disease in man or other animals, or any activity that otherwise affects the structure or any function of the body of man or other animals.

Therapeutic compounds may be administered to a subject in a variety of forms adapted to the chosen route of administration, e.g., orally or parenterally. Parenteral administration in this respect includes administration by the following routes: intravenous, intramuscular, subcutaneous, intraocular, intrasynovial, transepithelial, including transdermal, ophthalmic, sublingual and buccal; topically including ophthalmic, dermal, ocular, rectal and nasal inhalation via insufflation, aerosol and rectal systemic.

Compounds of Formula (I) and/or Formula (II) may be combined with a pharmaceutical carrier selected on the basis of the chosen route of administration and standard pharmaceutical practice. The relative proportions of active ingredient and carrier may be determined, for example, by the solubility and chemical nature of the compounds, chosen route of administration and standard pharmaceutical practice as would be understood by those skilled in the art. Therapeutic compounds may be administered by any means that may result in the contact of the active agent(s) with the desired site or site(s) of action in the body of a patient. The compounds may be administered by any conventional means available for use in conjunction with pharmaceuticals, either as individual therapeutic agents or in a combination of therapeutic agents. For example, they may be administered as the sole active agents in a pharmaceutical composition, or they can be used in combination with other therapeutically active ingredients.

In certain embodiments, the compound of Formula (I) can be co-administered with a compound of Formula (II), either in a single dosage form or separate dosage forms. In other embodiments, the compound of Formula (I) is administered prior to administration of a compound of Formula (II). In other embodiments, the compound of Formula (II) is administered prior to administration of a compound of Formula (I).

The compounds of the present disclosure can be formulated into any pharmaceutical dosage forms for oral, topical, rectal, vaginal, nasal, or ophthalmic administration, and include syrups and suspensions, and commonly known ingredients and procedures to formulate pharmaceutical composition are within the purview of a person skilled in the art, including various known methods can be used to formulate the composition of the invention.

### Examples

EXAMPLE 1. EFFECTS OF THE COMPOUND OF FORMULA (I) ON HYPERTHERMIA INDUCED BY THE COMPOUND OF FORMULA (II): Radio-telemetry methods are commonly used to study physiological functions related to regulation of body temperature and blood pressure. In laboratory rats, radio-telemetry probes can be used repeatedly for simultaneous recording of body temperature and blood pressure. As the compounds of Formula (I) and Formula (II), when given alone, produce hypotension (Delaunois, A. et al. J. Pharmacol. Toxicol. Meth., 2009, 60, 117) and hyperthermia (Honore, P. et al., Pain, 2009, 142, 27), respectively, radio-telemetry methods are applied to study body temperature and blood pressure effects of combinations of compounds of Formula (I) and Formula (II).

Adult male and female Sprague-Dawley are implanted with radio-telemetry transmitters for monitoring blood pressure and core body temperature. Radio-telemetry transmitter (DSI HD-S10) implantation is performed under sterile condition. Long-acting buprenorphine (1 mg/kg/day, SC) is administered prior to anesthesia for postoperative pain relief. Rats are anesthetized with 5% isoflurane and kept on 2% isoflurane to maintain a stable level of anesthesia. Body temperature is maintained during surgery using a heating pad. A laparotomy is performed. The catheter tip of the telemetry transmitter is secured with a 4-0 silk suture in the abdominal cavity. The body of the telemetry transmitter is placed in the abdominal cavity and secured to the abdominal wall. Approximately 100 μL of 0.25% marcaine is applied directly to the muscle incision, and the skin is then closed with a 5-0 Vicryl absorbable suture placed subcuticularly. After recovery from anesthesia, rats are returned to their home cages placed on DSI receivers.

After one week of recovery, rats are habituated to the drug administration procedure over three separate sessions. One week later, drugs tests are conducted. During these test sessions, the Dataquest A.R.T.™ acquisition and analysis system is utilized to monitor and analyze blood pressure (MAP, SBP, DBP), heart rate and core body temperature data in conscious, freely moving rats surgically implanted with radio-telemetry transmitters from 1 hour prior to dosing to up to 24 hours post dosing.

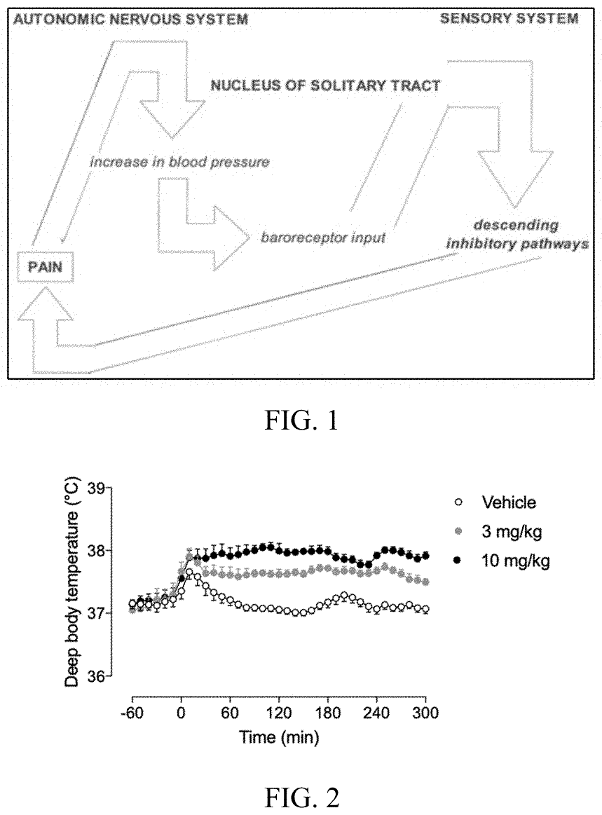

FIGS. 2-4 all show an increase in core body temperature approaching and at the zero time point. Those skilled in the art would readily appreciate that this increase is an artifact in the study associated with the required handling of the animals to effectuate the dosing.

FIG. 2 shows that administration of TRPV1 antagonist ABT-102 (3-10 mg/kg) (Axon Medchem) results in a dose dependent increase in core body temperature relative to vehicle. This effect is consistent with known effect of TRPV1 antagonism as reported in various clinical trials and preclinical studies, some of which are noted in the present background section.

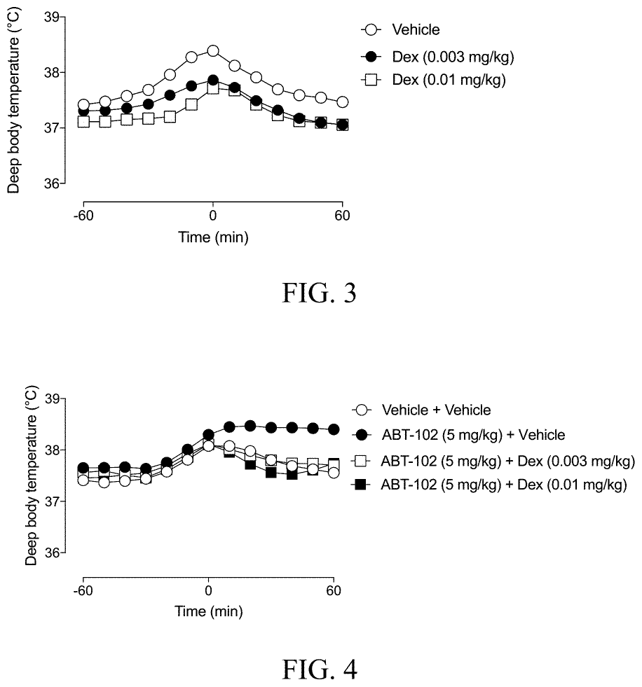

FIG. 3 illustrates that administration of alpha-2 agonist dexmedetomidine (Axon Medchem) has no effect on core body temperature.

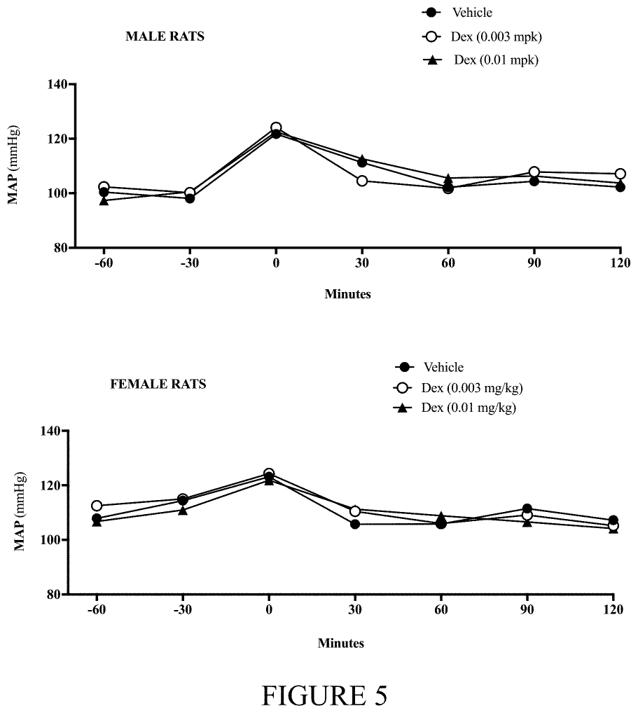

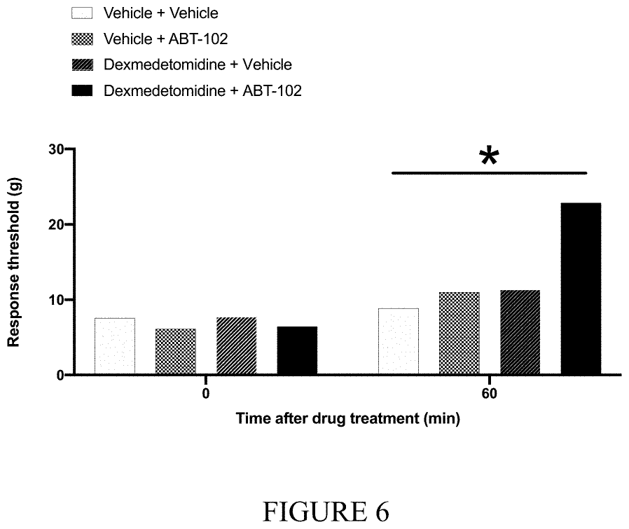

As shown in FIG. 4, when given in combination with ABT-102, dexmedetomidine prevented hyperthermic effects of the TRPV1 receptor antagonist (main overall effect of dexmedetomidine treatment, p<0.0001; time by treatment interaction, p<0.0001). Even at the lowest tested dose of 0.003 mg/kg, dexmedetomidine was able to neutralize ABT-102-induced hyperthermia. The observed attenuation is surprising given the lack of hypothermic effect illustrated in FIG. 3. Importantly, both 0.003 mg/kg and 0.01 mg/kg doses of dexmedetomidine were free from causing hypothermic effects on their own in both male and female rats as shown in FIG. 5. Accordingly, the reduction in hyperthermia is not attributable to deleterious hypotension.

EXAMPLE 2. ANTI-HYPERALGESIC AND ANTI-ALLODYNIC EFFECTS OF A COMBINATION OF THE COMPOUNDS OF FORMULA (I) AND FORMULA (II): Chronic constriction injury (CCI) models are commonly used to study neuropathic pain and to support discovery and development of novel therapeutics (Medvedev, I. O. et al., Neuropharmacology, 2004, 47, 175). Hyperalgesia and/or allodynia induced by CCI in laboratory rodents are sensitive to treatments aimed at α2-adrenoceptors (Murai, N. et al., Pharmacol. Biochem. Behav., 2016, 141, 10; Vissers, K. et al., Pain Practice, 2003, 3, 298) or TRPV1 receptors (Guo, S. H. et al., Sci. Rep., 2019, 9, 2769; Labuz, D. et al., Neuropharmacology, 2016, 101, 330). CCI models are used to reveal synergistic interactions of analgesic, anti-hyperalgesic and anti-allodynic, agents of different pharmacological classes. Lack of synergistic or supra-additive interactions is revealed using the CCI model for a combination of α2-adrenoceptor agonist with NMDA receptor channel blockers (Malyshkin, A. A. et al., Eur J Pharmacol, 2005, 519, 80).

Adult male and female Wistar rats with streptozotocin-induced experimental diabetes are anesthetized by isoflurane and two incisions are made—one on each thigh, parallel to the femoral bone and approximately 1.5 cm long. The common sciatic nerves are exposed by blunt dissection through biceps femoris on both sides. One paw is then designated as the “sham operated”. Contralateral paw is designated as “ligated”. The side of “ligated” paw is counterbalanced so that in half of the animals the left paw is “ligated”, whereas in another half of animals the left paw is “sham operated”. On the “ligated” paw side, proximal to the sciatic trifurcation four ligatures are tied loosely and spaced about 1 mm apart. The skin wounds are closed by 2-3 silk sutures.

After surgery, paw withdrawal tests are held on Days 4, 7 and 11 to monitor the development of tactile allodynia. Drug tests are administered twice a week starting from Day 14. Different doses are administered in a pseudorandom order. During tests, rats are placed into a plastic cage with a plastic string grid bottom, which allows full access to the paws. Short habituation period (5 min) precedes the test period. The paw withdrawal thresholds are determined as described before (Chaplan, S. R. et al., J. Pharmacol. Exp. Ther., 1994, 280, 829). Paws are touched with one of a series of 8 von Frey hairs with logarithmically incremental stiffness (0.692, 1.202, 2.041, 3.630, 5.495, 8.511, 15.136, 28.840 g). For each rat, the withdrawal thresholds on the left paw are always evaluated first followed by the same procedure on the right paw. The tip of the hair is presented perpendicular to the mid-plantar surface avoiding the less sensitive footpads. Sufficient force is applied to cause slight buckling against the paw, and is held for approximately 6-8 s. A positive response is noted either if the paw is sharply withdrawn or if flinching is seen immediately upon the removal of the hair. The testing is initiated with the 3.630 g hair. Stimuli are presented in consecutive fashion either ascending or descending. If no response is elicited by the initially selected hair (negative response), a stronger stimulus is presented. If the paw is withdrawn (positive response), the weaker stimulus is presented next. When the threshold is crossed, another 4 hairs in a same consecutive fashion are presented. All tests are performed by the experimenter blind to the treatment conditions.

The psychophysical 50% threshold is calculated using the up down method (Dixon, W. J., Ann. Rev. Pharmacol. Toxicol., 1980, 20, 441; Chaplan, S. R. et al., J. Pharmacol. Exp. Ther., 1994, 280, 829). For each animal the difference between paws is calculated by subtracting the log threshold value on the “ligated” paw from the log threshold value on the “sham-operated” paw (i.e., the positive values correspond to a lower threshold on the “ligated” paw).

Different doses of ABT-102 (3-30 mg/kg), dexmedetomidine (0.03-0.3 mg/kg), and their vehicles are administered one hour prior to the test to determine minimally and maximally effective doses as well as ED30 and ED50 values. Combined administration of submaximally effective doses of ABT-102 and dexmedetomidine is used to demonstrate supra-additive or synergistic interaction (FIG. 5). Two-way ANOVA with repeated measures has confirmed interaction between treatments (F(3,20)=6.28, P=0.0035). Post hoc analyses (Bonferroni's test) have suggested that effects of the ABT-102+dexmedetomidine combination were different from the vehicle+vehicle control group at 60-min post-treatment.

### Exemplary Embodiments

Clause 1. A pharmaceutical composition comprising, consisting of, or consisting essentially of an alpha-2 adrenoceptor agonist of Formula (I)

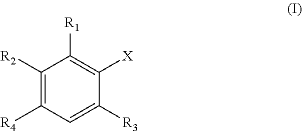

or a pharmaceutically acceptable salt or prodrug thereof, wherein

R₁ is H, Me, or Cl,

R₂ is H or Me, or

R₁ and R₂ together form

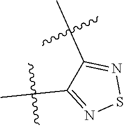 [SMILES: *c1nsnc1*]

R₃ is H, Me or Cl;

R₄ is H or tert-Bu;

X is selected from the group consisting of

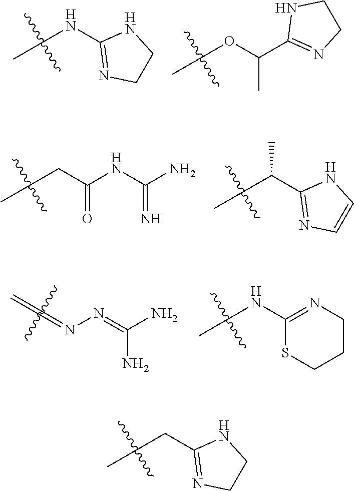

and a TRPV1 receptor antagonist of Formula (II):

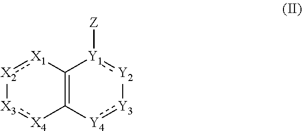

or a pharmaceutically acceptable salt or prodrug thereof, wherein [symbol]
is absent or single bond

### X₁ is CH, CMe, N or O;

X₂ is CH, CH₂, CNHAc or N;
X₃ is CR₁, (S)—CHCH₂OH, N, NH, or S;
X₄ is bond, CH or NR₂;

### R₁ is

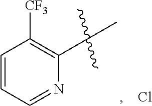 [SMILES: *c1ncccc1C(F)(F)F]

### R₂ is

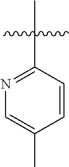 [SMILES: *c1ccc(C)cn1]

### Y₁ is C or N;

### Y₂ is CH, C═O or N;

Y₃ is CH or C-G, where G is a spiro-ring

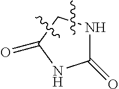

Y₄ is bond, CH or N;

### Z is

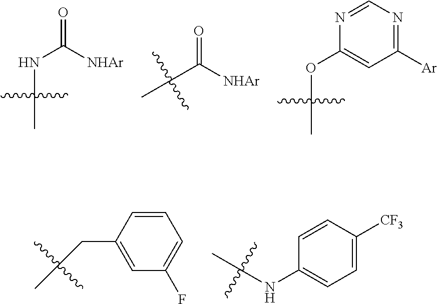

### Ar is

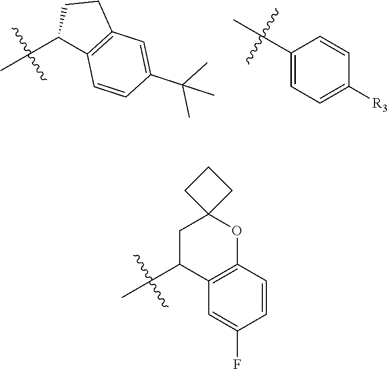

R₃ is CF₃ and OCH₂CF₃.

Clause 2. The pharmaceutical composition of clause 1, wherein the compound of Formula (I) is selected from the group consisting of clonidine, lofexidine, guanfacine, dexmedetomidine, guanabenz, tizanidine, brimonidine, xylazine and xylometazoline, an enantiomer thereof, a metabolite thereof, a derivative thereof, a prodrug thereof, a pharmaceutically acceptable salt thereof, or an acid addition salt or a combination thereof.

Clause 3. The pharmaceutical composition of clause 1 or 2, wherein the compound of Formula (I) is clonidine; or a pharmaceutically acceptable salt thereof; or an acid addition salt thereof.

Clause 4. The pharmaceutical composition of clause 1 or 2, wherein the compound of Formula (I) is lofexidine; or a pharmaceutically acceptable salt thereof; or an acid addition salt thereof.

Clause 5. The pharmaceutical composition of clause 1 or 2, wherein the compound of Formula (I) is guanfacine; or a pharmaceutically acceptable salt thereof; or an acid addition salt thereof.

Clause 6. The pharmaceutical composition of clause 1 or 2, wherein the compound of Formula (I) is dexmedetomidine; or a pharmaceutically acceptable salt thereof; or an acid addition salt thereof.

Clause 7. The pharmaceutical composition of clause 1 or 2, wherein the compound of Formula (I) is guanabenz; or a pharmaceutically acceptable salt thereof; or an acid addition salt thereof.

Clause 8, The pharmaceutical composition of clause 1 or 2, wherein the compound of Formula (I) is tizanidine; or a pharmaceutically acceptable salt thereof; or an acid addition salt thereof.

Clause 9. The pharmaceutical composition of clause 1 or 2, wherein the compound of Formula (I) is brimonidine; or a pharmaceutically acceptable salt thereof; or an acid addition salt thereof.

Clause 10. The pharmaceutical composition of clause 1 or 2, wherein the compound of Formula (I) is xylazine; or a pharmaceutically acceptable salt thereof; or an acid addition salt thereof.

Clause 11. The pharmaceutical composition of clause 1 or 2, wherein the compound of Formula (I) is xylometazoline; or a pharmaceutically acceptable salt thereof; or an acid addition salt thereof.

Clause 12. The pharmaceutical composition of clause 1 or 2, wherein the compound of Formula (I) is a combination of the compound as defined in clause 3 and the compound as defined in clause 4; the compound as defined in clause 3 and the compound as defined in clause 5; the compound as defined in clause 3 and the compound as defined in clause 6; the compound as defined in clause 3 and the compound as defined in clause 7; the compound as defined in clause 3 and the compound as defined in clause 8; the compound as defined in clause 3 and the compound as defined in clause 9; the compound as defined in clause 3 and the compound as defined in clause 10; the compound as defined in clause 3 and the compound as defined in clause 11; the compound as defined in clause 4 and the compound as defined in clause 5; the compound as defined in clause 4 and the compound as defined in clause 6; the compound as defined in clause 4 and the compound as defined in clause 7; the compound as defined in clause 4 and the compound as defined in clause 8; the compound as defined in clause 4 and the compound as defined in clause 9; the compound as defined in clause 4 and the compound as defined in clause 10; the compound as defined in clause 4 and the compound as defined in clause 11; the compound as defined in clause 5 and the compound as defined in clause 6; the compound as defined in clause 5 and the compound as defined in clause 7; the compound as defined in clause 5 and the compound as defined in clause 8; the compound as defined in clause 5 and the compound as defined in clause 9; the compound as defined in clause 5 and the compound as defined in clause 10; the compound as defined in clause 5 and the compound as defined in clause 11; the compound as defined in clause 6 and the compound as defined in clause 7; the compound as defined in clause 6 and the compound as defined in clause 8; the compound as defined in clause 6 and the compound as defined in clause 9; the compound as defined in clause 6 and the compound as defined in clause 10; the compound as defined in clause 6 and the compound as defined in clause 11; the compound as defined in clause 7 and the compound as defined in clause 8; the compound as defined in clause 7 and the compound as defined in clause 9; the compound as defined in clause 7 and the compound as defined in clause 10; the compound as defined in clause 7 and the compound as defined in clause 11; the compound as defined in clause 8 and the compound as defined in clause 9; the compound as defined in clause 8 and the compound as defined in clause 10; the compound as defined in clause 8 and the compound as defined in clause 11; the compound as defined in clause 9 and the compound as defined in clause 10; the compound as defined in clause 9 and the compound as defined in clause 11; or the compound as defined in clause 10 and the compound as defined in clause 11.

Clause 13. The pharmaceutical composition of any one of the preceding clauses, wherein the compound of Formula (II) is selected from the group consisting of N-(4-((6-(4-(trifluoromethyl)phenyl)pyrimidin-4-yl)oxy)benzo[d]thiazol-2-yl)acetamide, 5′-chloro-1′-(3-fluorobenzyl)-7′-methylspiro[imidazolidine-4,3′-indoline]-2,2′,5-trione, (R)-1-(5-(tert-butyl)-2,3-dihydro-1H-inden-1-yl)-3-(1H-indazol-4-yl)urea, (S)-3-(hydroxymethyl)-4-(5-methylpyridin-2-yl)-N-(4-(2,2,2-trifluoroethoxy)phenyl)-3,4-dihydro-2H-benzo[b][1,4]oxazine-8-carboxamide, (R)-1-(6-fluorospiro[chromane-2,1′-cyclobutan]-4-yl)-3-(isoquinolin-5-yl)urea, and N-(4-(trifluoromethyl)phenyl)-7-(3-(trifluoromethyl)pyridin-2-yl)quinazolin-4-amine; or an enantiomer thereof, a metabolite thereof, a derivative thereof, a prodrug thereof, a pharmaceutically acceptable salt thereof, or an acid addition salt or a combination thereof.

Clause 14. The pharmaceutical composition of any one of the preceding clauses, wherein the compound of Formula (II) is N-(4-((6-(4-(trifluoromethyl)phenyl)pyrimidin-4-yl)oxy)benzo[d]thiazol-2-yl)acetamide; or a pharmaceutically acceptable salt thereof; or an acid addition salt thereof.

Clause 15. The pharmaceutical composition of any one of the preceding clauses, wherein the compound of Formula (II) is 5′-chloro-1′-(3-fluorobenzyl)-7′-methylspiro[imidazolidine-4,3′-indoline]-2,2′,5-trione; or a pharmaceutically acceptable salt thereof; or an acid addition salt thereof.

Clause 16. The pharmaceutical composition of any one of the preceding clauses, wherein the compound of Formula (II) is (R)-1-(5-(tert-butyl)-2,3-dihydro-1H-inden-1-yl)-3-(1H-indazol-4-yl)urea; or a pharmaceutically acceptable salt thereof; or an acid addition salt thereof.

Clause 17. The pharmaceutical composition of any one of the preceding clauses, wherein the compound of Formula (II) is (S)-3-(hydroxymethyl)-4-(5-methylpyridin-2-yl)-N-(4-(2,2,2-trifluoroethoxy)phenyl)-3,4-dihydro-2H-benzo[b][1,4]oxazine-8-carboxamide; or a pharmaceutically acceptable salt thereof; or an acid addition salt thereof.

Clause 18. The pharmaceutical composition of any one of the preceding clauses, wherein the compound of Formula (II) is (R)-1-(6-fluorospiro[chromane-2,1′-cyclobutan]-4-yl)-3-(isoquinolin-5-yl)urea; or a pharmaceutically acceptable salt thereof; or an acid addition salt thereof.

Clause 19. The pharmaceutical composition of any one of the preceding clauses, wherein the compound of Formula (II) is N-(4-(trifluoromethyl)phenyl)-7-(3-(trifluoromethyl)pyridin-2-yl)quinazolin-4-amine; or a pharmaceutically acceptable salt thereof; or an acid addition salt thereof.

Clause 20. The pharmaceutical composition of any one of the preceding claims, wherein the compound of Formula (II) is the combination of the compound as defined in clause 14 and the compound as defined in clause 15; the compound as defined in clause 14 and the compound as defined in clause 16; the compound as defined in clause 14 and the compound as defined in clause 17; the compound as defined in clause 14 and the compound as defined in clause 18; the compound as defined in clause 14 and the compound as defined in clause 19; the compound as defined in clause 15 and the compound as defined in clause 16; the compound as defined in clause 15 and the compound as defined in clause 17; the compound as defined in clause 15 and the compound as defined in clause 18; the compound as defined in clause 15 and the compound as defined in clause 19; the compound as defined in clause 16 and the compound as defined in clause 17; the compound as defined in clause 16 and the compound as defined in clause 18; the compound as defined in clause 16 and the compound as defined in clause 19; the compound as defined in clause 17 and the compound as defined in clause 18; the compound as defined in clause 17 and the compound as defined in clause 19; the compound as defined in clause 18 and the compound as defined in clause 19.

Clause 21. A method of treatment of pain, comprising administering a therapeutically effective amount of the composition of any one of the preceding clauses to a patient in need thereof.

Clause 22. The method of clause 21, wherein pain is acute pain.

Clause 23. The method of clause 21, wherein the pain is neuropathic pain.

Clause 24. The method of clause 21, wherein the pain is cancer pain.

Clause 25. The method of clause 21, wherein the pain is dental pain.

Clause 25A. The method of clause 21, wherein the pain is postoperative pain.

Clause 26. The method of clause 21, wherein the pain is associated with an inflammatory bowel disorder or inflammatory pain.

Clause 27. The method of clause 21, wherein the pain is associated with an inflammatory eye disorder.

Clause 28. The method of clause 21, wherein the pain is skin pain associated with inflammation.

Clause 29. The method of clause 21, wherein the pain is chronic pain; and optionally, wherein the chronic pain is selected from the group consisting of somatogenic, neurogenic, and psychogenic pain.

Clause 30. The method of clause 21, wherein the pain is associated with hyperalgesia or allodynia, where the hyperalgesia or allodynia is optionally induced by: a disease or disease state known to cause hyperalgesia or allodynia; a medication; an illicit drug; or a therapeutic or recreational drug.

Clause 31. The method of clause 21, wherein the pain is the combination of any two or more pain conditions as defined in clauses 22 to 30.

Clause 32. A method of reducing a TRPV1 antagonist-induced increase in body temperature in a mammal comprising administering an alpha-2 adrenoceptor agonist to the mammal, preferably, a human.

Clause 33. A method of treating pain by a composition comprising administering a TRPV1 antagonist in combination with alpha-2 agonist, which reduces the risk of TRPV1 antagonist-induced hyperthermia, to the mammal, preferably, a human.

Clause 34. A method of treating pain by a composition comprising administering a TRPV1 antagonist in combination with alpha-2 agonist, which reduces the risk of alpha-2 agonist-induced hypotension, to the mammal, preferably, a human.

Clause 35. The method as defined in clause 32, 33, or 34, wherein the alpha-2 agonist is a compound of Formula (I) is a compound as defined in any one of clauses 1 to 12.

Clause 36. A method of any one of the clauses 21-35, wherein the TRPV1 antagonist and an alpha-2 agonist; or compounds of Formulas I and II are formulated as a single composition for administration to the human subjects.

Clause 37. A method according any one of the clauses 21-36, wherein the alpha-2 agonist is administered up to 3 hours before, simultaneously, or up to 3 hours after administration of TRPV1 antagonist.

Clause 38. A method of decreasing the number of doses and/or total daily dose of the compound of Formula (II) by administering the compound of Formula (II) as defined in any one of clauses 1 and 13-20 in combination with a compound of Formula (I) as defined in any one of clauses 1 to 12.

Clause 39. A method of decreasing the number of doses and/or total daily dose of the compound of Formula (I) by administering the compound of Formula (I) as defined in any one of clauses 1 to 12 in combination with a compound of Formula (II) as defined in any one of clauses 1 and 13-20.

Clause 40. The method according to any one of clauses 21-39, wherein the administration is cutaneous, oral, nasal, rectal, vaginal, sublingual, buccal, sublabial, muscular, intramuscular, intravenous, intraperitoneal or peritoneal, epidural, intracerebral, intracerebroventricular, epicutaneous or topical, intraarticular, intracardiac, intracavernous, intradermal, intralesional, intraocular, intraosseous, intrathecal, intrauterine, intravaginal, intravesical, intravitreal, transdermal, or transmucosal.

Clause 41. A pharmaceutical composition comprising a one or more agents, each having at least one unique Therapeutic Mode of Action (TMA), wherein the agent is TRPV1 receptor antagonist, and/or α2-adrenoceptor agonist, wherein the agent is a compound as defined in any one of clauses 1 to 20.

Clause 42. The pharmaceutical composition according to clause 41, wherein the content of agent in the composition is between 0.01 mg and 1000 mg.

Clause 43. The method according to any one of clauses 21 to 40, wherein the patient, mammal, or human is hypertensive.

Clause 44. A method of treating pain, comprising administering to a patient in need thereof the composition of clause 41 or 42.

Clause 45. The composition as defined in any one of clauses 1 to 20 or 42 to 43 for use in the treatment of pain.

Clause 46. Use of the composition as defined in any one of clauses 1 to 20 or 42 to 43 for the manufacture of a medicament for the treatment of pain.

Clause 47. The composition as defined in clause 45 or the use of clause 46 wherein pain is as defined in any one of clauses 22 to 31.

We claim:

1. A pharmaceutical composition comprising alpha-2 adrenoceptor agonist of Formula (I) 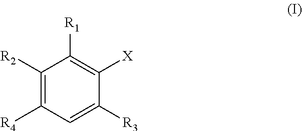
or a pharmaceutically acceptable salt or prodrug thereof, wherein
R₁ is H, Me, or Cl,
R₂ is H or Me, or
R₁ and R₂ together form 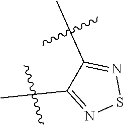 [SMILES: *c1nsnc1*]
R₃ is H, Me or Cl;
R₄ is H or tert-Bu;
X is selected from the group consisting of 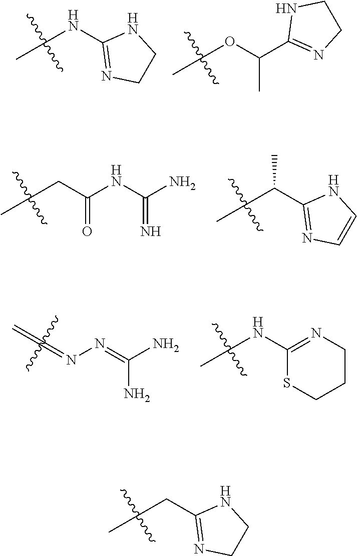
and a TRPV1 receptor antagonist of Formula (II): 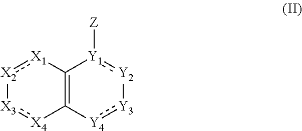
or a pharmaceutically acceptable salt or prodrug thereof, wherein [symbol]
is absent or single bond
X₁ is CH, CMe, N or O;
X₂ is CH, CH₂, CNHAc or N;
X₃ is CR₁, (S)—CHCH₂OH, N, NH, or S;
X₄ is bond, CH or NR₂;
R₁ is 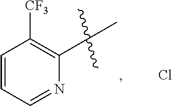 [SMILES: *c1ncccc1C(F)(F)F ; Cl]
R₂ is 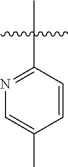 [SMILES: *c1ccc(C)cn1]
Y₁ is C or N;
Y₂ is CH, C═O or N;
Y₃ is CH or C-G, where G is a spiro-ring 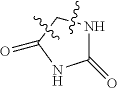
Y₄ is bond, CH or N;
Z is 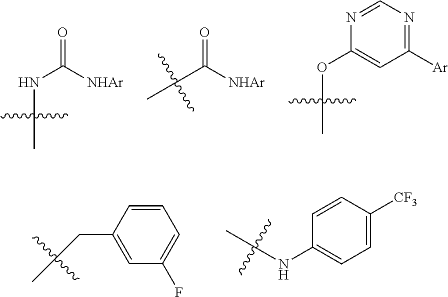
Ar is 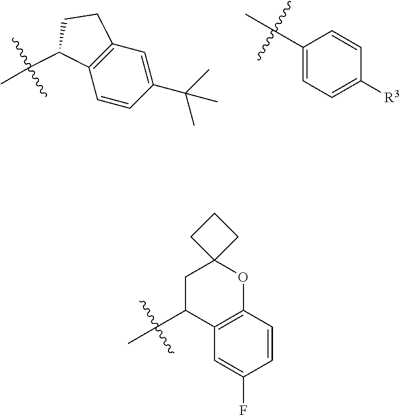
R₃ is CF₃ and OCH₂CF₃.

2. The pharmaceutical composition of claim 1, wherein the compound of Formula (I) is selected from the group consisting of clonidine, lofexidine, guanfacine, dexmedetomidine, guanabenz, tizanidine, brimonidine, xylazine and xylometazoline, an enantiomer thereof, a metabolite thereof, a derivative thereof, a prodrug thereof, a pharmaceutically acceptable salt thereof, or an acid addition salt or a combination thereof.

3. The pharmaceutical composition of claim 1, wherein the compound of Formula (II) is selected from the group consisting of N-(4-((6-(4-(trifluoromethyl)phenyl)pyrimidin-4-yl)oxy)benzo[d]thiazol-2-yl)acetamide, 5′-chloro-1′-(3-fluorobenzyl)-7′-methylspiro[imidazolidine-4,3′-indoline]-2,2′,5-trione, (R)-1-(5-(tert-butyl)-2,3-dihydro-1H-inden-1-yl)-3-(1H-indazol-4-yl)urea, (S)-3-(hydroxymethyl)-4-(5-methylpyridin-2-yl)-N-(4-(2,2,2-trifluoroethoxy)phenyl)-3,4-dihydro-2H-benzo[b][1,4]oxazine-8-carboxamide, (R)-1-(6-fluorospiro[chromane-2,1′-cyclobutan]-4-yl)-3-(isoquinolin-5-yl)urea, and N-(4-(trifluoromethyl)phenyl)-7-(3-(trifluoromethyl)pyridin-2-yl)quinazolin-4-amine; or an enantiomer thereof, a metabolite thereof, a derivative thereof, a prodrug thereof, a pharmaceutically acceptable salt thereof, or an acid addition salt or a combination thereof.

4. A method of treating pain comprising administering a TRPV1 antagonist and an alpha-2 adrenoreceptor agonist to a patient in need thereof.

5. The method of claim 4, wherein the TRPV1 antagonist is a compound of Formula (II): 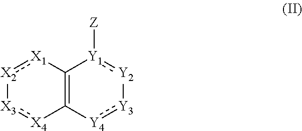
or a pharmaceutically acceptable salt or prodrug thereof, wherein [symbol]
is absent or single bond
X₁ is CH, CMe, N or O;
X₂ is CH, CH₂, CNHAc or N;
X₃ is CR₁, (S)—CHCH₂OH, N, NH, or S;
X₄ is bond, CH or NR₂;
R₁ is 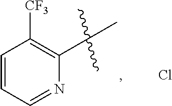 [SMILES: *c1ncccc1C(F)(F)F ; Cl]
R₂ is 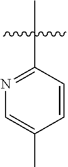 [SMILES: *c1ccc(C)cn1]
Y₁ is C or N;
Y₂ is CH, C═O or N;
Y₃ is CH or C-G, where G is a spiro-ring 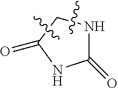
Y₄ is bond, CH or N;
Z is 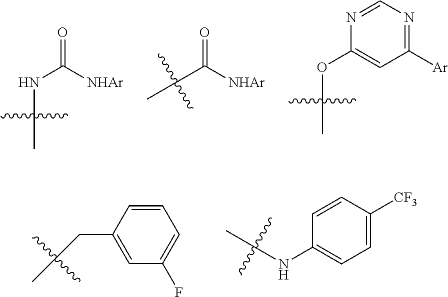
Ar is 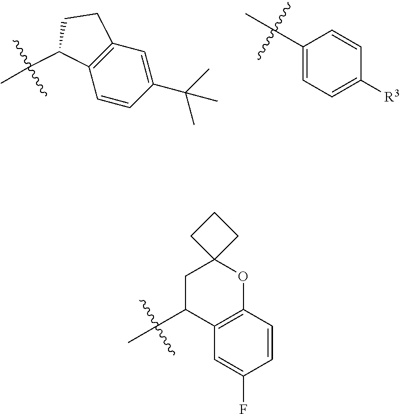
R₃ is CF₃ and OCH₂CF₃.

6. The method of claim 5, wherein the TRPV1 antagonist is selected from the group consisting of N-(4-((6-(4-(trifluoromethyl)phenyl)pyrimidin-4-yl)oxy)benzo[d]thiazol-2-yl)acetamide, 5′-chloro-1′-(3-fluorobenzyl)-7′-methylspiro[imidazolidine-4,3′-indoline]-2,2′,5-trione, (R)-1-(5-(tert-butyl)-2,3-dihydro-1H-inden-1-yl)-3-(1H-indazol-4-yl)urea, (S)-3-(hydroxymethyl)-4-(5-methylpyridin-2-yl)-N-(4-(2,2,2-trifluoroethoxy)phenyl)-3,4-dihydro-2H-benzo[b][1,4]oxazine-8-carboxamide, (R)-1-(6-fluorospiro[chromane-2,1′-cyclobutan]-4-yl)-3-(isoquinolin-5-yl)urea, and N-(4-(trifluoromethyl)phenyl)-7-(3-(trifluoromethyl)pyridin-2-yl)quinazolin-4-amine; or a pharmaceutically acceptable salt thereof, or an acid addition salt or a combination thereof.

7. The method of claim 4, wherein the alpha-2 agonist is a compound of Formula (I): 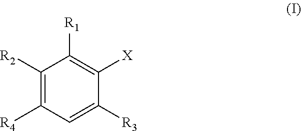
or a pharmaceutically acceptable salt or prodrug thereof, wherein
R₁ is H, Me, or Cl,
R₂ is H or Me, or
R₁ and R₂ together form 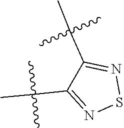 [SMILES: *c1nsnc1*]
R₃ is H, Me or Cl;
R₄ is H or tert-Bu;
X is selected from the group consisting of

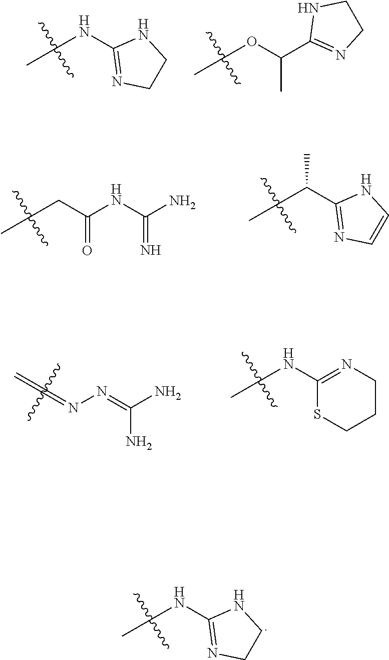

8. The method of claim 7, wherein the alpha-2 agonist is selected from the group consisting of clonidine, lofexidine, guanfacine, dexmedetomidine, guanabenz, tizanidine, brimonidine, xylazine and xylometazoline; or a pharmaceutically acceptable salt thereof, or an acid addition salt or a combination thereof.

9. The method of claim 4, wherein the TRPV1 antagonist and an alpha-2 agonist are formulated as a single pharmaceutical composition.

10. The method of claim 4, wherein the alpha-2 agonist is administered up to 3 hours before, simultaneously, or up to 3 hours after administration of TRPV1 antagonist.

11. The method of claim 4, wherein pain is acute pain.

12. The method of claim 4, wherein the pain is neuropathic pain.

13. The method of claim 4, wherein the pain is cancer pain.

14. The method of claim 4, wherein the pain is dental pain.

15. The method of claim 4, wherein the pain is associated with an inflammatory bowel disorder.

16. The method of claim 4, wherein the pain is associated with an inflammatory eye disorder.

17. The method of claim 4, wherein the pain is skin pain associated with inflammation.

18. The method of claim 4, wherein the pain is inflammatory pain.

19. The method of claim 4, wherein the pain is associated with hyperalgesia or allodynia.

20. A method of treating pain, comprising administering the pharmaceutical composition of claim 1 to a patient in need thereof.
